(19) United States
(12) Patent Application Publication
(10) Pub. No.: US 2026/0226548 A1
(43) Pub. Date: Aug. 6, 2026

(54) NOVEL ASSAY FOR PHASING OF DISTANT GENOMIC LOCI WITH ZYGOSITY RESOLUTION VIA LONG-READ SEQUENCING HYBRID DATA ANALYSIS

(71) Applicant: F. HOFFMANN-LA ROCHE AG, Basel (CH)

(72) Inventors: Szymon Tomasz Calus, Lörrach (DE); Nicolas Giroud, Basel (CH); Anna Leena Rautanen, Basel (CH); Manuel Ignacio Araujo Novoa, Basel (CH)

(73) Assignee: F. HOFFMANN-LA ROCHE AG, Basel (CH)

(21) Appl. No.: 19/152,659

(22) PCT Filed: Mar. 28, 2024
(86) PCT No.: PCT/EP2024/058424

§ 371 Date: Jul. 31, 2025

(30) Foreign Application Priority Data
Mar. 31, 2023 (EP) 23165992.1

(51) Int. Cl. C12Q 1/6883; C12N 15/113; C12Q 1/6858

(52) U.S. Cl. CPC C12Q 1/6883; C12N 15/113; C12Q 1/6858; C12N 2310/11; C12N 2320/34; C12Q 2600/118; C12Q 2600/156; C12Q 2600/172

(57) ABSTRACT

The present invention describes a novel method for pre-clinical or clinical biomarker characterization, for example in the field of neuroscience (e.g. Huntington's disease). The methods and kits disclosed may be used as a companion diagnostic tool, where identification of two or more paired loci are needed to provide essential information for a safe and efficient stratification of a patient with a specific therapy or drug treatment. More particularly, the method allows for accurate assignment of the spatial relationship of a single nucleotide polymorphism (SNP) with a short-tandem repeat (STR) or another SNP from a highly distant region in a genome at a hetero/homozygosity resolution.

### FIELD OF THE INVENTION

The present disclosure provides a novel molecular biology assay for pre-clinical or clinical biomarker characterization, for example in the field of neuroscience (e.g. Huntington's disease, HD). The methods and kits disclosed may be used as a companion diagnostic tool, where identification of two or more paired loci are needed to provide essential information for a safe and efficient stratification of a patient with a specific therapy or drug treatment.

### BACKGROUND OF THE INVENTION

Huntington's disease (HD) is a rare genetic disorder that causes a progressive degeneration of brain cells. HD disorder is caused by an abnormal expansion of short tandem repeats (STR) in the huntingtin gene chromosome 4, exon 1. These repeats, consisting of a sequence of cytosine-adenine-guanine (CAG) nucleotides. A normal huntingtin gene typically has 10-35 CAG repeats, but in individuals with Huntington's disease, the number of CAG repeats can range from 36 to over 100. The CAG expansions can occur spontaneously or be inherited in an autosomal dominant manner. If a parent has a CAG expansion in the huntingtin gene, there is a 50% chance of passing it on to their children. Individuals with a CAG expansion (commonly of more than 40 repeat units) have a 100% risk of developing Huntington's disease during their lifetime. As the number of CAG repeats increases, the age of onset of symptoms decreases and the severity of the disease increases. The exact mechanism by which CAG expansions cause Huntington's disease is not fully understood, but it is thought that the expanded CAG repeats cause abnormal folding of the huntingtin protein, leading to the accumulation of toxic protein aggregates that damage brain cells, leading to cell death. Irregular expansion in STR's (such as CAG repeat expansions) are not unique to Huntington's disease (HD) and have been associated with several other genetic disorders, including spinocerebellar ataxias, myotonic dystrophy, and several forms of muscular dystrophy (Hannan, 2018). Several SNPs have been identified in the huntingtin gene that modify the risk of developing Huntington's disease, as well as with variations in the age of onset and severity of symptoms (Claassen, D. O. et al., 2020; Bečanović, K. et al., 2015). For example, several SNPs in the huntingtin gene known as rs13102260, rs362277, rs3025814, rs2530596 have been associated with the age of onset (Kartsaki, E., et al., 2006; Kay, C., et al. 2015; Ramos, E. M. et al., 2012). Moreover, certain SNP alleles at one polymorphic site (such as, e.g., rs7685686, rs362331, rs6446723, rs6844859, rs363080s rs363125, rs362307, rs362273) have been identified to enable selective treatment of HD patients while also allowing the possibility of nonselective treatment of all remaining patients (Kay, C., et al. 2015; Shin et al. 2022). Such intronic and exonic SNPs may be targeted using antisense oligonucleotide (ASO) based therapies to selectively target precursor mRNA (pre-mRNA) or messenger RNA (mRNA) to alter the mRNA and thereby protein expression through a variety of mechanisms.

Advances in long-read sequencing technologies, such as sequencing platforms based on long-read technology commercially available, e.g., from Oxford Nanopore Technologies Ltd. (Oxford, UK) and Pacific Biosciences of California, Inc. (Menlo Park, USA), have made it possible to accurately detect and characterize SNPs, as well as CAG repeat expansions, in the huntingtin gene and other genes associated with Huntington's disease. The identification of SNPs associated with Huntington's disease and related disorders is an important area of research, as it may enable the development of new approaches to disease management and treatment. For example, SNPs may provide targets for the development of new drugs or therapies that can modulate the expression or activity of the huntingtin protein, or for the development of gene-editing approaches that can modify the huntingtin gene to prevent or reverse disease progression. Overall, the identification and characterization of SNPs associated with Huntington's disease is an important area of research with the potential to improve our understanding of the disease and to advance the development of new treatments and therapies. The Huntington condition manifests itself as a loss of GABAergic medium spiny (GABA MS) neurons in the striatum and caused by an expansion of the CAG repeat in exon 1 of the huntingtin gene. Within cells, this wild-type protein may be involved in chemical signaling, transporting materials, attaching (binding) to proteins and other structures, and protecting the cell from self-destruction (apoptosis). The symptoms of Huntington's disease usually start to appear in middle age, but they can also occur earlier or later in life. The early symptoms include involuntary movements, such as jerking or twitching, as well as difficulty with coordination and balance. As the disease progresses, the symptoms become more severe and can include cognitive impairment, mood swings, and behavioral changes. The time from the first symptoms to death is often about 10 to 30 years. There is no cure, but treatment can alleviate symptoms and support is available. Huntingtin protein is found in many of the body's tissues (e.g. liver), with the highest levels of activity in the brain. Additionally, genetic counseling and testing can help individuals and families understand their risk of developing the disease and make informed decisions about family planning.

DNA sequencing is a fundamental tool in biological and medical research, and is especially important for the paradigm of personalized medicine. Various new DNA sequencing methods have been investigated with the aim of eventually realizing the goal of the $1,000 genome; the dominant method is sequencing by synthesis (SBS), an approach that determines short DNA sequences during the polymerase reaction (Slatko et al., 2018). PacBio sequencing, also referred to as SMRT (Single-Molecule Real-Time) sequencing, enables very long fragments to be sequenced, up to 30-50 kb. The SMRT method involves binding an engineered DNA polymerase to the bottom of a Zero-Mode Waveguides (ZMWs) well, where the DNA library ligated with SMRT-bell adapters is loaded onto the DNA polymerase. The four nucleotides are labeled with different phospho-linked fluorophores for differential detection. When a nucleotide is incorporated into the growing chain, imaging occurs on the millisecond time scale as the correct fluorescently-labeled nucleotide is incorporated into a complementary strain of the single stranded DNA molecule sequenced. After each dNTP polymerization process, the phosphate-linked fluorescent moiety is released and dissipates from the detection region and can no longer be detected. The next nucleotide can then be incorporated. Herein, imaging is timed with the rate of nucleotide incorporation so that each base is identified as it is incorporated into the growing DNA chain. (Slatko et al., above). Nanopore-based DNA sequencing was first proposed in the late 1990s and commercialization has recently been achieved by Oxford Nanopore Technologies Ltd. (Oxford, UK), wherein protein nanopores are embedded into an electrically resistant bilayer membrane through which characteristic changes in electrical current (picoampere—pA) occur as each nucleotide passes through the detector allowing short, long and ultra-long read lengths, from 0.1 kb up to 1 Mb. More specifically, long dsDNA molecules are first ligated to a motor enzyme and tether molecule, which allow the libraries to sediment onto the sequencing flow cell and increase proximity of the molecule to the nanopore and by the same increases the amount of molecules available for analysis. When the guide strand containing a motor-complex encounters an available nanopore, the template of single stranded DNA (ssDNA) enters the nanopore and disrupts the pA baseline readout via alteration. Herein, the translocation rate is regulated by nucleotide sequence and accompanying epigenetic modifications. The motor enzyme enables the DNA to slow down processivity through the channel and increases the quality of the raw data. Each nucleotide k-mer present in the nanopore provides a characteristic electronic pattern that is recorded in real time as a current disruption event (Slatko et al., above) and is subsequently base called into a standardized FASTQ/FASTA dataset.

Well established short-read Sequencing-by-Synthesis (SBS) platforms (such as, e.g., MiSeq or NextSeq Series by Illumina, Inc., San Diego, CA, USA or Ion GeneStudio Systems by Thermo Fisher, Waltham, MA, USA) are routinely used for SNP genotyping and there are several algorithms for resolving haplotypes based on the SNP data. However, such approaches are limited in many aspects as they are mainly designed for haplotyping of whole genome assemblies and rely on existing population based reference panels. This is a statistical approach, which is never 100% accurate and more complex genetic variants such as STRs are not included in the reference panels. Also, regions of low genetic variation would lead to breaking of the phasing information between distant loci. Novel sequencing platforms based on long-read technology (available, e.g., from Oxford Nanopore Technologies Ltd., Oxford, UK and Pacific Biosciences of California, Inc., Menlo Park, USA) can help resolving the SNP phasing directly without statistical inference but the high error rate of Oxford Nanopore sequencing and shorter mean read length (~20 kb) of PacBio are a limitation in deconvolution of >150 kb distant regions at single nucleotide level in an accurate, low cost and fast manner. To overcome the problem of phasing distant SNPs together with the CAG repeat, Asuragen, Inc. (Austin, TX, USA) is using AmplideX® PCR technology to develop companion diagnostic tests that size and phase HTT CAG repeats with two different SNPs targeted by Wave's WVE-120101 and WVE-120102 investigational therapeutic programs. However, their approach of using RNA as a starting material to reduce the distance between the CAG repeat and exonic SNPs, cannot be used for intronic SNPs. Alternative methods for phasing of distant SNP with CAG repeats could include oligo-based hybridization capture of a full-length gene via biotinylated probes along the sequence of interest and subsequent PCR amplification (such as, e.g., #101341 Twist Custom Panel Plus or #102989 Twist Human Custom Comprehensive Exome available, from Twist Biosciences, South San Francisco, US). This method requires genomic DNA material as input and whole genome amplification followed by enrichment step. However, extraction of very long genomic DNA fragments (e.g. 15-30 kb nt) is still difficult and the approach may lead to fragmentation of the enriched material and loss of desired long signal needed for phasing of distant loci, especially for the regions with low genetic variation. Another alternative method of phasing two distant loci is provided in WO2018/022473 comprising the amplification of two genomic regions each comprising a locus of interest using primer sets leading to sticky-end amplification products, which are subsequently subjected to a ligation procedure. Herein, one type of nucleic acid is used as template for determining the loci of interest (e.g., chromosome or a fragment thereof, genomic DNA or mRNA/cDNA). Due to random ligation of the amplifications products, the resulting ligation products cannot directly be used for haplotyping. Consequently, the method disclosed in WO2018/022473 requires partitioning the nucleic acid template into droplets, so that only one nucleic acid molecule template is present per droplet. PCR amplification of the two different loci and sticky-end ligation are performed in the droplets. Subsequently, a second amplification reaction of the properly ligated product is required before sequencing the amplified ligated product by next-generation sequencing. Thus, this method is very laborious and complex and due to the many handling steps is prone to phasing errors and artefacts. WO 2016/191380 describes the general idea of phasing SNPs from long-read sequencing data, using heterozygous SNPs as reference for aligning the sequences from one type of nucleic acid (i.e., DNA or RNA). This method relies on having several heterozygous SNPs in a locus if long distances need to be covered by the sequencing run. However, depending on the target sequence to be analyzed and the positions of the SNPs used for phasing, this may not always be possible.

Therefore, a need exists for a novel method, which overcomes such technical issues related to the necessity for high-input of genomic DNA, short-reads, custom oligonucleotide development or high-error profile.

### SUMMARY OF THE INVENTION

In order to tackle the shortcomings of current methods, the present disclosure provides a hybrid analysis of long amplicons generated from genomic DNA and cDNA (mRNA) for pairing of distant information via spatial linkage, i.e., phasing of two or more required loci. Such methods may provide for significant advancements in applied diagnostics and are a prerequisite for allele-specific treatment for genetic diseases such as Huntington's disease. Applying a hybrid approach as disclosed herein using both DNA and cDNA (reverse transcribed from mature mRNA) as a starting material, it is possible to phase a short tandem repeat or an exonic single nucleotide polymorphism of interest and a distant target SNP (intronic or exonic) via an exonic reference SNP using only a very small number of amplicons (e.g., at least one DNA and at least one cDNA amplicon). Herein, the method allows for the analysis of a heterozygous and a homozygous exonic or intronic target SNP. In particular, in order to best resolve the phasing information, the exonic reference SNP should be heterozygous if the target SNP is heterozygous.

The methods and kits are particularly applicable in the field of companion diagnostics, which involves the identification of loci to provide critical information on a patient's genomic status that can be used to safely and effectively match a patient with a specific treatment or drug therapy thereby identifying and stratifying patients who are most likely to benefit from a particular drug treatment or therapy. Such applications have become increasingly important in recent years as more targeted therapies are being developed. Furthermore, the methods and kits of the disclosure may also be used in basic research, where it can aid in the identification of genetic variations associated with various diseases and conditions. This can lead to a better understanding of the underlying mechanisms of diseases, which can ultimately lead to the development of new therapies and treatments.

In one aspect, a method of phasing at least one distant single nucleotide polymorphism of interest and short tandem repeats within the same target gene locus of nucleic acids isolated from a biological sample is provided, the method comprising: (a) performing an amplification step comprising contacting genomic DNA isolated from said biological sample comprising the target gene locus with a first set of oligonucleotide primers to produce a first amplification product comprising the at least one single nucleotide polymorphism of interest and at least one exonic reference single nucleotide polymorphism in the same gene locus; (b) performing a reverse transcription and amplification step comprising contacting mRNA isolated from said biological sample comprising the target gene locus with a second set of oligonucleotide primers to produce a second amplification product comprising the short tandem repeats and said at least one exonic reference single nucleotide polymorphism; (c) determining the nucleic acid sequence of the first and the second amplification product; (d) aligning the nucleic acid sequences of the first and the second amplification product determined in step (c) based on the position of the at least one exonic reference single nucleotide polymorphism; and (e) determining the haplotype of the at least one distant single nucleotide polymorphism of interest and the short tandem repeats in the sample. In one embodiment, the at least one distant single nucleotide polymorphism of interest is an intronic single nucleotide polymorphism. In a particular embodiment, the intron carrying the at least one distant single nucleotide polymorphism of interest is located adjacent to the exon carrying the at least one exonic reference single nucleotide polymorphism within the same gene locus of the isolated genomic DNA. In one embodiment, the amplification step (a) and the reverse transcription and amplification step (b) are performed in separate reaction vessels. In another embodiment, the nucleic acid sequence of the first and the second amplification product in step (c) is determined using long-read sequencing, such as PacBio sequencing, also referred to as SMRT (Single Molecule Real Time) sequencing, or nanopore-based DNA long-read sequencing as commercialized by Oxford Nanopore Technologies. In another embodiment, the biological sample is selected from the group consisting of tissue, tumor tissue, blood, saliva and cell lines derived from an individual. In one embodiment, when aligning the nucleic acid sequences of the first and the second amplification product in step (d) the at least one exonic reference single nucleotide polymorphism is heterozygous if the at least one distant single nucleotide polymorphism of interest is heterozygous. In another embodiment, prior to aligning the nucleic acid sequences of the first and the second amplification product in step (d) the method further comprises the step of aligning the nucleic acid sequence of the first and the second amplification product to the nucleic acid sequence of a reference gene of the target gene locus or to the nucleic acid sequence of the complete human genome or to parts thereof comprising a reference gene of target gene locus. In another embodiment, the target gene locus is the huntingtin gene. In a particular embodiment, the short tandem repeats are CAG repeats. In some embodiments, the at least one single nucleotide polymorphism of interest is selected from the group consisting of rs7685686, rs362331, rs363088, rs362273, rs2024115, rs6446723, rs363064, rs2285086, rs6844859, rs363080, rs34315806, rs2298967, rs362271, rs363099, rs3121419, and rs16843804. In certain embodiments, the at least one single nucleotide polymorphism of interest is an intronic SNP selected from the group consisting of rs7685686, rs363088, rs2024115, rs6446723, rs363064, rs2285086, rs6844859, rs363080, rs2298967, rs362271, rs3121419, and rs16843804. In certain embodiments, the at least one single nucleotide polymorphism of interest is an exonic SNP selected from the group consisting of rs362331, rs362273, rs34315806 and rs363099. In a particular embodiment, the at least one single nucleotide polymorphism of interest is intronic SNP rs7685686. In some embodiments, the at least one exonic reference single nucleotide polymorphism is selected from the group consisting of rs362331, rs362273, rs34315806, and rs363099. Herein, the exonic reference single nucleotide polymorphism (such as, e.g., rs362331, rs362273, rs34315806, and rs363099) must not be the same as (i.e., does not correspond to) the at least one single nucleotide polymorphism of interest. In some embodiments, the at least one exonic reference single nucleotide polymorphism is selected from the group consisting of rs362331, rs362273, rs34315806, and rs363099 if the at least one single nucleotide polymorphism of interest is an intronic SNP. In certain embodiments, the at least one exonic reference single nucleotide polymorphism is rs362331. In certain embodiments, determining the haplotype of the short tandem repeats comprises determining the number of the short tandem repeats units. In certain embodiments, the mRNA is a spliced mRNA (mature mRNA).

In another aspect, an in vitro method for diagnosing if an individual has a risk of developing Huntington's disease is provided, the method comprising: (a) determining the haplotype of at least one distant single nucleotide polymorphism of interest and short tandem repeats in an in vitro sample obtained from said individual by performing an amplification step comprising contacting genomic DNA isolated from said biological sample comprising the target gene locus with a first set of oligonucleotide primers to produce a first amplification product comprising the at least one single nucleotide polymorphism of interest and at least one exonic reference single nucleotide polymorphism in the same gene locus; performing a reverse transcription and amplification step comprising contacting mRNA isolated from said biological sample comprising the target gene locus with a second set of oligonucleotide primers to produce a second amplification product comprising the short tandem repeats and said at least one exonic reference single nucleotide polymorphism; determining the nucleic acid sequence of the first and the second amplification product; aligning the nucleic acid sequences of the first and the second amplification product determined in the previous step based on the position of the at least one exonic reference single nucleotide polymorphism; and determining the haplotype of the at least one distant single nucleotide polymorphism of interest and the short tandem repeats in the sample; (b) determining the individual's risk of developing Huntington's disease based on the haplotype of the at least one distant single nucleotide polymorphism of interest and the CAG short tandem repeats determined in step (a). In one embodiment, the at least one distant single nucleotide polymorphism of interest is an intronic single nucleotide polymorphism. In a particular embodiment, the intron carrying the at least one distant single nucleotide polymorphism of interest is located adjacent to the exon carrying the at least one exonic reference single nucleotide polymorphism within the same gene locus of the isolated genomic DNA. In one embodiment, the amplification step and the reverse transcription and amplification step are performed in separate reaction vessels. In another embodiment, the nucleic acid sequence of the first and the second amplification product is determined using long-read sequencing, such as PacBio sequencing, also referred to as SMRT (Single Molecule Real Time) sequencing, or nanopore-based DNA long-read sequencing as commercialized by Oxford Nanopore Technologies. In another embodiment, the biological sample is selected from the group consisting of tissue, tumor tissue, blood, saliva and cell lines derived from an individual. In one embodiment, when aligning the nucleic acid sequences of the first and the second amplification product the at least one exonic reference single nucleotide polymorphism is heterozygous if the at least one distant single nucleotide polymorphism of interest is heterozygous. In another embodiment, prior to aligning the nucleic acid sequences of the first and the second amplification product the method further comprises the step of aligning the nucleic acid sequence of the first and the second amplification product to the nucleic acid sequence of a reference gene of the target gene locus or to the nucleic acid sequence of the complete human genome or to parts thereof comprising a reference gene of target gene locus. In another embodiment, the target gene locus is the huntingtin gene. In a particular embodiment, the short tandem repeats are CAG repeats. In certain embodiments, the at least one single nucleotide polymorphism of interest is an intronic SNP selected from the group consisting of rs7685686, rs363088, rs2024115, rs6446723, rs363064, rs2285086, rs6844859, rs363080, rs2298967, rs362271, rs3121419, and rs16843804. In certain embodiments, the at least one single nucleotide polymorphism of interest is an exonic SNP selected from the group consisting of rs362331, rs362273, rs34315806 and rs363099. In a particular embodiment, the at least one single nucleotide polymorphism of interest is intronic SNP rs7685686. In some embodiments, the at least one exonic reference single nucleotide polymorphism is selected from the group consisting of rs362331, rs362273, rs34315806, and rs363099. Herein, the exonic reference single nucleotide polymorphism (such as, e.g., rs362331, rs362273, rs34315806, and rs363099) must not be the same as (i.e., does not correspond to) the at least one single nucleotide polymorphism of interest. In some embodiments, the at least one exonic reference single nucleotide polymorphism is selected from the group consisting of rs362331, rs362273, rs34315806, and rs363099 if the at least one single nucleotide polymorphism of interest is an intronic SNP. In certain embodiments, the at least one exonic reference single nucleotide polymorphism is rs362331. In certain embodiments, determining the haplotype of the short tandem repeats comprises determining the number of the short tandem repeats units. In certain embodiments, the mRNA is a spliced mRNA (mature mRNA).

In another aspect, an in vitro method of identifying a patient having Huntington's disease as likely to respond to a therapy targeting at least one single nucleotide polymorphism of interest, the method comprising: (a) determining the haplotype of the at least one distant single nucleotide polymorphism of interest and short tandem repeats in an in vitro sample obtained from said individual by performing an amplification step comprising contacting genomic DNA isolated from said biological sample comprising the target gene locus with a first set of oligonucleotide primers to produce a first amplification product comprising the at least one single nucleotide polymorphism of interest and at least one exonic reference single nucleotide polymorphism in the same gene locus; performing a reverse transcription and amplification step comprising contacting mRNA isolated from said biological sample comprising the target gene locus with a second set of oligonucleotide primers to produce a second amplification product comprising the short tandem repeats and said at least one exonic reference single nucleotide polymorphism; determining the nucleic acid sequence of the first and the second amplification product; aligning the nucleic acid sequences of the first and the second amplification product determined in the previous step based on the position of the at least one exonic reference single nucleotide polymorphism; and determining the haplotype of the at least one distant single nucleotide polymorphism of interest and the short tandem repeats in the sample; (b) identifying the patient as likely to respond to the therapy based on the haplotype determined in step (a) and the determination of the presence of a specific allele of the at least one single nucleotide polymorphism of interest. In some embodiments, the therapy targeting at least one single nucleotide polymorphism of interest is an antisense oligonucleotide treatment directed to suppress an RNA molecule comprising the specific allele of the at least one single nucleotide polymorphism of interest. In one embodiment, the at least one distant single nucleotide polymorphism of interest is an intronic single nucleotide polymorphism. In a particular embodiment, the intron carrying the at least one distant single nucleotide polymorphism of interest is located adjacent to the exon carrying the at least one exonic reference single nucleotide polymorphism within the same gene locus of the isolated genomic DNA. In one embodiment, the amplification step (a) and the reverse transcription and amplification step (b) are performed in separate reaction vessels. In another embodiment, the nucleic acid sequence of the first and the second amplification product in step (c) is determined using long-read sequencing, such as PacBio sequencing, also referred to as SMRT (Single Molecule Real Time) sequencing, or nanopore-based DNA long-read sequencing as commercialized by Oxford Nanopore Technologies. In another embodiment, the biological sample is selected from the group consisting of tissue, tumor tissue, blood, saliva and cell lines derived from an individual. In one embodiment, when aligning the nucleic acid sequences of the first and the second amplification product the at least one exonic reference single nucleotide polymorphism is heterozygous if the at least one distant single nucleotide polymorphism of interest is heterozygous. In another embodiment, prior to aligning the nucleic acid sequences of the first and the second amplification product the method further comprises the step of aligning the nucleic acid sequence of the first and the second amplification product to the nucleic acid sequence of a reference gene of the target gene locus or to the nucleic acid sequence of the complete human genome or to parts thereof comprising a reference gene of target gene locus. In another embodiment, the target gene locus is the huntingtin gene. In a particular embodiment, the short tandem repeats are CAG repeats. In some embodiments, the at least one single nucleotide polymorphism of interest is selected from the group consisting of rs7685686, rs362331, rs363088, rs362273, rs2024115, rs6446723, rs363064, rs2285086, rs6844859, rs363080, rs34315806, rs2298967, rs362271, rs363099, rs3121419, and rs16843804. In certain embodiments, the at least one single nucleotide polymorphism of interest is an intronic SNP selected from the group consisting of rs7685686, rs363088, rs2024115, rs6446723, rs363064, rs2285086, rs6844859, rs363080, rs2298967, rs362271, rs3121419, and rs16843804. In certain embodiments, the at least one single nucleotide polymorphism of interest is an exonic SNP selected from the group consisting of rs362331, rs362273, rs34315806 and rs363099. In a particular embodiment, the at least one single nucleotide polymorphism of interest is intronic SNP rs7685686. In some embodiments, the at least one exonic reference single nucleotide polymorphism is selected from the group consisting of rs362331, rs362273, rs34315806, and rs363099. Herein, the exonic reference single nucleotide polymorphism (such as, e.g., rs362331, rs362273, rs34315806, and rs363099) must not be the same as (i.e., does not correspond to) the at least one single nucleotide polymorphism of interest. In some embodiments, the at least one exonic reference single nucleotide polymorphism is selected from the group consisting of rs362331, rs362273, rs34315806, and rs363099 if the at least one single nucleotide polymorphism of interest is an intronic SNP. In certain embodiments, the at least one exonic reference single nucleotide polymorphism is rs362331. In certain embodiments, determining the haplotype of the short tandem repeats comprises determining the number of the short tandem repeats units. In certain embodiments, the mRNA is a spliced mRNA (mature mRNA).

In another aspect, a kit for determining the nucleic acid sequence of at least one distant single nucleotide polymorphism of interest and short tandem repeats within the same target gene locus of nucleic acids isolated from a biological sample is provided, the kit comprising a first set of oligonucleotide primers to produce a first amplification product comprising the at least one single nucleotide polymorphism of interest and at least one exonic reference single nucleotide polymorphism in the same gene locus; and a second set of oligonucleotide primers to produce a second amplification product comprising the at least one exonic single nucleotide polymorphism of interest and said at least one exonic reference single nucleotide polymorphism. Herein, the kit is adapted for performing any of the methods disclosed herein. In a particular embodiment, the first set of oligonucleotide primers comprises an oligonucleotide primer comprising the nucleic acid sequence of SEQ ID NO:1 and an oligonucleotide primer comprising the nucleic acid sequence of SEQ ID NO:2. In another particular embodiment, the second set of oligonucleotide primers comprises an oligonucleotide primer comprising the nucleic acid sequence of SEQ ID NO:3, an oligonucleotide primer comprising the nucleic acid sequence of SEQ ID NO:4 and an oligonucleotide primer comprising the nucleic acid sequence of SEQ ID NO:5. In some embodiments, the kit further includes at least one of nucleoside triphosphates, nucleic acid polymerase, and buffers necessary for the function of the nucleic acid polymerase and/or a reverse transcriptase. In some embodiments, the kit further includes any one of reagents for amplification, such as a reverse transcriptase, a DNA polymerase, dNTPs, buffers, and/or other elements (e.g., cofactors or aptamers) appropriate for reverse transcription and/or amplification. Typically, the reagent mixture(s) is concentrated, so that an aliquot is added to the final reaction volume, along with sample (e.g., RNA or DNA), enzymes, and/or water. In some embodiments, the kit further comprises reverse transcriptase (or an enzyme with reverse transcriptase activity), and/or DNA polymerase (e.g., thermostable DNA polymerase such as Taq, ZO5, and derivatives thereof).

In another aspect, a method of phasing at least one distant single nucleotide polymorphism of interest and at least one exonic single nucleotide polymorphism of interest within the same target gene locus of nucleic acids isolated from a biological sample, the method comprising: (a) performing an amplification step comprising contacting genomic DNA isolated from said biological sample comprising the target gene locus with a first set of oligonucleotide primers to produce a first amplification product comprising the at least one single nucleotide polymorphism of interest and at least one exonic reference single nucleotide polymorphism in the same gene locus; (b) performing a reverse transcription and amplification step comprising contacting mRNA isolated from said biological sample comprising the target gene locus with a second set of oligonucleotide primers to produce a second amplification product comprising the at least one exonic single nucleotide polymorphism of interest and said at least one exonic reference single nucleotide polymorphism; (c) determining the nucleic acid sequence of the first and the second amplification product; (d) aligning the nucleic acid sequences of the first and the second amplification product determined in step (c) based on the position of the at least one exonic reference single nucleotide polymorphism; and (e) determining the haplotype of the at least one distant single nucleotide polymorphism of interest and the at least one exonic single nucleotide polymorphism of interest in the sample. In one embodiment, the at least one distant single nucleotide polymorphism of interest is an intronic single nucleotide polymorphism. In a particular embodiment, the intron carrying the at least one distant single nucleotide polymorphism of interest is located adjacent to the exon carrying the at least one exonic reference single nucleotide polymorphism within the same gene locus of the isolated genomic DNA. In one embodiment, the amplification step (a) and the reverse transcription and amplification step (b) are performed in separate reaction vessels. In another embodiment, the nucleic acid sequence of the first and the second amplification product in step (c) is determined using long-read sequencing, such as PacBio sequencing, also referred to as SMRT (Single Molecule Real Time) sequencing, or nanopore-based DNA long-read sequencing as commercialized by Oxford Nanopore Technologies. In another embodiment, the biological sample is selected from the group consisting of tissue, tumor tissue, blood, saliva and cell lines derived from an individual. In one embodiment, when aligning the nucleic acid sequences of the first and the second amplification product in step (d) the at least one exonic reference single nucleotide polymorphism is heterozygous if the at least one distant single nucleotide polymorphism of interest is heterozygous. In another embodiment, prior to aligning the nucleic acid sequences of the first and the second amplification product in step (d) the method further comprises the step of aligning the nucleic acid sequence of the first and the second amplification product to the nucleic acid sequence of a reference gene of the target gene locus or to the nucleic acid sequence of the complete human genome or to parts thereof comprising a reference gene of target gene locus. In another embodiment, the target gene locus is the huntingtin gene. In some embodiments, the at least one single nucleotide polymorphism of interest is selected from the group consisting of rs7685686, rs362331, rs363088, rs362273, rs2024115, rs6446723, rs363064, rs2285086, rs6844859, rs363080, rs34315806, rs2298967, rs362271, rs363099, rs3121419, and rs16843804. In certain embodiments, the at least one single nucleotide polymorphism of interest is an intronic SNP selected from the group consisting of rs7685686, rs363088, rs2024115, rs6446723, rs363064, rs2285086, rs6844859, rs363080, rs2298967, rs362271, rs3121419, and rs16843804. In certain embodiments, the at least one single nucleotide polymorphism of interest is an exonic SNP selected from the group consisting of rs362331, rs362273, rs34315806 and rs363099. In a particular embodiment, the at least one single nucleotide polymorphism of interest is intronic SNP rs7685686. In some embodiments, the at least one exonic reference single nucleotide polymorphism is selected from the group consisting of rs362331, rs362273, rs34315806, and rs363099. Herein, the exonic reference single nucleotide polymorphism (such as, e.g., rs362331, rs362273, rs34315806, and rs363099) must not be the same as (i.e., does not correspond to) the at least one single nucleotide polymorphism of interest. In some embodiments, the at least one exonic reference single nucleotide polymorphism is selected from the group consisting of rs362331, rs362273, rs34315806, and rs363099 if the at least one single nucleotide polymorphism of interest is an intronic SNP. In certain embodiments, the at least one exonic reference single nucleotide polymorphism is rs362331. In certain embodiments, the mRNA is a spliced mRNA (mature mRNA).

In another aspect, an antisense oligonucleotide specifically hybridizing to at least one distant single nucleotide polymorphism of interest in an huntingtin (HTT) gene for use for treating a patient having Huntington's disease is provided, wherein the patient is selected for treatment when determining a specific haplotype of the at least one distant single nucleotide polymorphism of interest and the CAG short tandem repeats detected in a biological sample of the patient. In some embodiments, the at least one distant single nucleotide polymorphism of interest is selected from the group consisting of rs7685686, rs362331, rs363088, rs362273, rs2024115, rs6446723, rs363064, rs2285086, rs6844859, rs363080, rs34315806, rs2298967, rs362271, rs363099, rs3121419, and rs16843804. In certain embodiments, the at least one single nucleotide polymorphism of interest is an intronic SNP selected from the group consisting of rs7685686, rs363088, rs2024115, rs6446723, rs363064, rs2285086, rs6844859, rs363080, rs2298967, rs362271, rs3121419, and rs16843804. In certain embodiments, the at least one single nucleotide polymorphism of interest is an exonic SNP selected from the group consisting of rs362331, rs362273, rs34315806 and rs363099. In a particular embodiment, the at least one distant single nucleotide polymorphism of interest is rs7685686. In some embodiments, the specific haplotype comprises the presence of the A allele for the rs7685686, and the number of CAG short tandem repeats is above 36. In other embodiments, the specific haplotype comprises the presence of the G allele for the rs7685686, and the number of CAG short tandem repeats is above 36. In some embodiments, the antisense oligonucleotide specifically hybridizes to a region in the huntingtin (HTT) gene comprising the A allele for the rs7685686. In some embodiments, the antisense oligonucleotide is selected to specifically hybridize to the distant single nucleotide polymorphism of interest in an allele specific fashion. In some embodiments, the specific haplotype is determined using the methods disclosed herein.

In another aspect, an in vitro use of haplotype determination of at least one distant single nucleotide polymorphism of interest and the short tandem repeats detected in the huntingtin (HTT) gene determined in a biological sample of an individual for diagnosing Huntington's disease is provided, wherein the detection of a specific haplotype of the at least one distant single nucleotide polymorphism of interest and the short tandem repeats detected in a biological sample of the patient indicates that the individual has disease Huntington's disease. In some embodiments, the at least one single nucleotide polymorphism of interest is selected from the group consisting of rs7685686, rs362331, rs363088, rs362273, rs2024115, rs6446723, rs363064, rs2285086, rs6844859, rs363080, rs34315806, rs2298967, rs362271, rs363099, rs3121419, and rs16843804. In certain embodiments, the at least one single nucleotide polymorphism of interest is an intronic SNP selected from the group consisting of rs7685686, rs363088, rs2024115, rs6446723, rs363064, rs2285086, rs6844859, rs363080, rs2298967, rs362271, rs3121419, and rs16843804. In certain embodiments, the at least one single nucleotide polymorphism of interest is an exonic SNP selected from the group consisting of rs362331, rs362273, rs34315806 and rs363099. In a particular embodiment, the at least one single nucleotide polymorphism of interest is intronic SNP rs7685686. In some embodiments, the specific haplotype comprises the presence of the A allele for the rs7685686, and the number of CAG short tandem repeats is above 36. In other embodiments, the specific haplotype comprises the presence of the G allele for the rs7685686, and the number of CAG short tandem repeats is above 36. In some embodiments, the specific haplotype is determined using the methods disclosed herein.

In another aspect, an in vitro use of haplotype determination of at least one distant single nucleotide polymorphism of interest and the short tandem repeats detected in the huntingtin (HTT) gene determined in a biological sample of a patient having from Huntington's disease for determining the patient as likely to respond to a therapy comprising an antisense oligonucleotide specifically hybridizing to at least one distant single nucleotide polymorphism of interest in an huntingtin (HTT) gene, wherein the patient is identified as being more likely to respond to the therapy when a specific haplotype of the at least one distant single nucleotide polymorphism of interest and the short tandem repeats is detected in a biological sample of the patient. In some embodiments, the at least one single nucleotide polymorphism of interest is selected from the group consisting of rs7685686, rs362331, rs363088, rs362273, rs2024115, rs6446723, rs363064, rs2285086, rs6844859, rs363080, rs34315806, rs2298967, rs362271, rs363099, rs3121419, and rs16843804. In certain embodiments, the at least one distant single nucleotide polymorphism of interest is selected from the group consisting of rs7685686, rs362331, rs363088, rs362273, rs2024115, rs6446723, rs363064, rs2285086, rs6844859, rs363080, rs34315806, rs2298967, rs362271, rs363099, rs3121419, and rs16843804. In certain embodiments, the at least one single nucleotide polymorphism of interest is an intronic SNP selected from the group consisting of rs7685686, rs363088, rs2024115, rs6446723, rs363064, rs2285086, rs6844859, rs363080, rs2298967, rs362271, rs3121419, and rs16843804. In certain embodiments, the at least one single nucleotide polymorphism of interest is an exonic SNP selected from the group consisting of rs362331, rs362273, rs34315806 and rs363099. In a particular embodiment, the at least one single nucleotide polymorphism of interest is intronic SNP rs7685686. In some embodiments, the specific haplotype comprises the presence of the A allele for the rs7685686, and the number of CAG short tandem repeats is above 36. In some embodiments, the antisense oligonucleotide specifically hybridizes to a region in the huntingtin (HTT) gene comprising the A allele for the rs7685686. In other embodiments, the specific haplotype comprises the presence of the G allele for the rs7685686, and the number of CAG short tandem repeats is above 36. In some embodiments, the antisense oligonucleotide is selected to specifically hybridize to the distant single nucleotide polymorphism of interest in an allele specific fashion. In some embodiments, the specific haplotype is determined using the methods disclosed herein.

Further disclosed is a method of treating a patient having Huntington's disease, the method comprising: (a) determining the haplotype of at least one distant single nucleotide polymorphism of interest and the short tandem repeats in the huntingtin (HTT) gene in a biological sample of the patient; and (b) administering an antisense oligonucleotide specifically hybridizing to the at least one distant single nucleotide polymorphism of interest in an huntingtin (HTT) gene. Herein, the at least one distant single nucleotide polymorphism of interest may be selected from the group consisting of rs7685686, rs362331, rs363088, rs362273, rs2024115, rs6446723, rs363064, rs2285086, rs6844859, rs363080, rs34315806, rs2298967, rs362271, rs363099, rs3121419, and rs16843804. In certain embodiments, the at least one single nucleotide polymorphism of interest is an intronic SNP selected from the group consisting of rs7685686, rs363088, rs2024115, rs6446723, rs363064, rs2285086, rs6844859, rs363080, rs2298967, rs362271, rs3121419, and rs16843804. In certain embodiments, the at least one single nucleotide polymorphism of interest is an exonic SNP selected from the group consisting of rs362331, rs362273, rs34315806 and rs363099. In a particular embodiment, the at least one single nucleotide polymorphism of interest is intronic SNP rs7685686. Also disclosed is that the haplotype as determined comprises the presence of the A allele for the rs7685686, and the number of CAG short tandem repeats is above 36. Also disclosed is that the antisense oligonucleotide specifically hybridizes to a region in the huntingtin (HTT) gene comprising the A allele for the rs7685686. In other embodiments, the specific haplotype comprises the presence of the G allele for the rs7685686. In some embodiments, the antisense oligonucleotide is selected to specifically hybridize to the distant single nucleotide polymorphism of interest in an allele specific fashion. Herein, the specific haplotype may be determined using the methods disclosed herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 indicates the distant separation between tandem-repeat region (i.e. CAG) and intronic or exonic SNP of interest. Distance is being represented on genomic DNA level and may span up to 200 kb nucleotides. The intronic and exonic SNP are separated from each other by only around 10 kb and can be amplified with a PCR assay (i.e. F1 and R1 primers). The transcription and mRNA maturation of the gene target involves splicing and thereby reduction of the distance between CAG tandem repeats and exonic reference SNP, which allows for reverse transcription and PCR of a ~10 kb amplicon from a single molecule (i.e. F2 and R2 primers).

FIG. 2 depicts two amplicons i.e. first generated from a genomic DNA and containing the intronic target SNP and exonic reference SNP; second generated from cDNA derived from mature mRNA and containing CAG tandem repeats and exonic reference SNP. The hybrid analysis allows for phasing of distant intronic SNP with a number of CAG tandem repeats by an alignment of exonic SNP between two amplicons.

FIG. 3 shows the phasing results for the sample GM04282. FIG. 3A The cDNA data indicates that mutant HTT allele with ~77.6 CAGs (“High CAG”) comprising ~22.7% of the reads, shows a phasing with the exonic T allele in ~95% of the reads. The wild-type HTT allele with ~18.1 CAG's (“Low CAG”) comprising ~77.2% of the reads also shows ~95% of the reads having exonic T allele. Both alleles have >4% of reads with undefined nucleotides at the position of the SNP of interest, this may be due to sequencing errors such as deletions. As the exonic SNP is homozygous, it is not informative for the hybrid analysis. FIG. 3B The bar plots of the cDNA data indicate presence of two distributions (i.e. low and high CAG repeat number) for mean values in a range of ~18 and ~77 repeats (FIG. 3A). The exonic SNP allele for both distributions is specified as Thymine (FIG. 3A), indicating homozygosity. FIG. 3C The DNA amplicon data indicates that the exonic reference SNP allele T is phased in 100% of the passed reads with the intronic target SNP allele A. Other alleles have not been detected indicating homozygosity of the intronic target SNP for high and low CAG repeat regions. Therefore, this patient would not be suitable for allele-specific treatment.

FIG. 4 shows the phasing results for the sample GM13503. FIG. 4A The cDNA data indicates that mutant HTT allele with ~46.3 CAGs (“High CAG”) comprising ~48.1% of the reads, shows a phasing with the exonic T allele in ~99.3% of the reads and only 0.7% for C allele. While the wild-type HTT allele with 18 CAG's (“Low CAG”) comprising ~51.9% of reads and shows ~99.7% of the reads having exonic C allele and only 0.16% for T allele. Both alleles have close to 0% of reads with undefined nucleotides at the position of the SNP of interest. As the exonic SNP is heterozygous, this patient could be suitable for allele-specific treatment, however further identification of intronic SNP being phased with high CAG is needed. FIG. 4B The bar plots of the cDNA data indicate two distributions (i.e. low and high CAG number) for mean values in a range of ~18 and ~46 repeats (FIG. 4A). The exonic SNP for high CAG number is specified as Thymine (FIG. 4A) while low CAG allele has majority of reads assigned as Cytosine, indicating heterozygosity of the exonic reference SNP. FIG. 4C The DNA amplicon data indicates that the exonic reference SNP allele C is phased in >98% of the reads with the intronic target SNP allele G and only 1.33% with allele A. While the exonic reference SNP allele T is phased in ~98.03% of the reads with intronic target SNP allele A and only 1.97% with the intronic allele G. The low percentage of the outlying phasing results (1.33% and 1.97%) can be a result of PCR-errors or chimeric molecules, which in turn results in small noise in a final dataset. As a conclusion, the mutant CAG allele (high CAG) is phased with exonic reference SNP allele T, which in turn is phased with intronic target SNP allele A, making this patient eligible for A specific ASO treatment.

FIG. 5 shows the phasing results for the sample GM04724. FIG. 5A The cDNA data indicates that mutant HTT allele with ~71.2 CAGs (“High CAG”) comprising ~30.7% of the reads, shows a phasing with the reference exonic SNP T allele in ~96.68% of the reads and only ~0.55% for C allele. While the wild-type HTT allele with ~16 CAG's (“Low CAG”) comprising ~69.3% of reads shows ~98.16% of the reads having exonic C allele and only 0.25% for T allele. Two alleles indicate 1.59% and 2.77% of reads with undefined nucleotides at the position of the SNP of interest; this may be due to errors such as insertions or deletions. As the exonic SNP is heterozygous, this patient could be suitable for allele-specific treatment, however further identification of intronic SNP being phased with high CAG is needed. FIG. 5B The bar plots of the cDNA data indicate two distributions (i.e. low and high CAG number) for mean values in a range of ~16 and ~71.2 repeats (FIG. 5A). The exonic SNP allele for high CAG number is specified as Thymine (FIG. 5A) while low CAG allele has majority of reads assigned as Cytosine, indicating heterozygosity of the exonic reference SNP. FIG. 5C The DNA amplicon data indicates that the exonic reference SNP allele C is phased in 97.54% of the reads with the intronic target SNP allele G and only 2.46% with allele A. While the exonic reference SNP allele T is phased in 98.8% of the reads with the intronic, target SNP allele A and only 1.2% with the intronic allele G. The low percentage of the outlying phasing results (2.46% and 1.2%) can be a result of PCR-errors or chimeric molecules, which in turn results in small noise in a final dataset. As a conclusion, the mutant CAG allele (high CAG) is phased with exonic reference SNP allele T, which in turn is phased with intronic target SNP allele A, making this patient eligible for A specific ASO treatment as in the example shown in FIG. 4.

FIG. 6 Depicts the outcome of clustering reads with a similarity threshold of 1.0 (i.e., clusters formed by identical reads). Outcomes represent two final contigs showing the presence or absence of the variant alleles of the intronic and exonic SNP's. Residual reads, which did not cluster into main haplotype groups, were omitted from the analysis due to presence of nucleotides with low quality scores, which resulted in sporadic errors at various nucleotide positions and lack of 100% similarity between core cluster off reads.

FIG. 7 represents an outcome from the Integrative Genomics Viewer (IGV, Broad Institute) software for two final DNA amplicon clusters, including exonic and intronic SNP positions for heterozygous samples.

FIG. 8 shows a flow chart of the bioinformatics analysis, starting from the raw sequencing data until visualization of the DNA/cDNA reads and phasing of CAG number with intronic SNP.

FIG. 9 shows a detailed flow chart of the bioinformatics analysis, starting from the raw sequencing data until visualization of the DNA/cDNA reads and phasing of CAG number with intronic SNP.

FIG. 10 shows a scheme for using the phase information of the instant methods for stratification and selection of patients likely to respond to SNP specific antisense oligonucleotide treatment.

### DETAILED DESCRIPTION OF THE INVENTION

### I. Introduction

Phasing of short tandem repeats (STRs) together with distant single nucleotide polymorphisms (SNPs) within the same gene locus at individual patient level is challenging. Standard methodologies such as PCR followed by short-read sequencing of genomic DNA are not suitable for such analyses if the genetic variants are too far away from each other (more than tens of kb). To overcome this problem, long-range PCR using mRNA as a starting material for the PCR amplification followed by long-read sequencing helps to bring exonic SNPs closer to distant STRs. This approach, however, is not suitable for intronic SNPs. Thus, there remains a need in the art for a reliable method to provide haplotype information for STRs and distant SNPs, particularly intronic SNPs, within the same gene locus. Moreover, there also remains a need for a reliable method to provide haplotype information for one or more exonic SNPs and one or more distant SNPs, particularly intronic SNPs, within the same gene locus.

To tackle this task, the present disclosure provides a novel molecular biology assay for pre-clinical or clinical biomarker characterization, for example in the field of neuroscience (e.g. Huntington's disease, HD). Such methods may be used as a companion diagnostic tool, where identification of two or more paired loci are needed to provide essential information for a safe and efficient stratification of a patient with a specific therapy or drug treatment. More particularly, the method allows for accurate assignment of spatial relationship (i.e. phase/ing) of a single nucleotide polymorphism (SNP) with a short-tandem repeat (STR) or another SNP from a highly distant region in a genome (e.g. >150 kb) at a haploid genome level. Method is based on extraction of DNA and RNA and parallel amplification of DNA and cDNA obtained from a single sample (e.g. blood or tissue) with subsequent hybrid analysis of data provided by a long-read sequencing technology such as Pacific Biosciences of California, Inc. or Oxford Nanopore Technologies Limited.

Herein, methods are provided that analyze samples to phase exonic and/or intronic SNP alleles together with STRs or other distant SNPs. In certain aspects, methods are provided that analyze samples to phase exonic and/or intronic SNP alleles together with a trinucleotide CAG repeat region that is located more than 150 kb away and use the phase information for stratification of patients (e.g., for inclusion of patients in clinical trials or subjecting patients to therapeutic treatment). In particular aspects, phasing information for the trinucleotide CAG repeat region and at least one distant SNP (such as, e.g., rs7685686) within the huntingtin gene may be used to determine whether or not a patient may benefit from antisense oligonucleotide (ASO) treatment targeting the particular distant SNP for Huntington's disease.

The present invention provides several advantages over current methods for SNP and STR genotyping. Most importantly, this novel method allows for the accurate determination of the spatial relationship between SNPs and STRs or other SNPs from highly distant regions in the genome at haploid genome level, which is not possible with the standard SNP and STR genotyping methods. This is particularly important for the allele-specific treatment approaches. The goal of the allele-specific treatment of, e.g., Huntington's disease is to use SNP allele specific antisense oligonucleotide (ASOs) to suppress the mutant (expanded CAG) RNA molecule and keep the wild type intact. Therefore the assay described herein is crucial to identify which SNP allele is in the same haplotype (i.e., in phase) with the expanded CAG repeat.

Comprehensive design of PCR primers is usually required to develop a robust and precise assay. Problems may appear in non-specific amplification of random gene fragments, which would increase signal to noise in the final sequencing data. However, alignment to the human reference genome sequence allows only the target sequence to be considered in the final analysis. Moreover, the design of the PCR primers and the hybrid assay must take in account the linkage disequilibrium (LD) between intronic target SNP and exonic reference SNPs in order to maximize the informativeness of the fingerprinting approach (if either of the SNPs is homozygote, for example allele-specific treatment is not possible). LD structure varies between different populations and that has to be taken into account in the assay design.

The workflow for the most difficult scenario, in which the target SNP is intronic and too far away from the CAG repeat to be amplified from genomic DNA with PCR is as follows:
(1) Co-extraction of DNA and RNA from a single sample.
(2) RT-PCR amplification of the region to cover the CAG repeat and the reference exonic SNP using RNA as an input material to bring the exonic reference SNP closer to the CAG repeat. The reference exonic SNP should ideally be in high linkage disequilibrium with the target intronic SNP or at least in the same allele-frequency range.
(3) PCR amplification of the region to cover the above-mentioned reference exonic SNP and the intronic target SNP using genomic DNA as an input material.
(4) Sequencing of the amplicons using long-read technology to ensure that the phasing information between the variants within the amplicon is maintained.
(5) Hybrid data-analysis to align the DNA and cDNA amplicon sequences and phase the intronic target SNP allele with the CAG repeat number via using exonic reference SNP fingerprint.

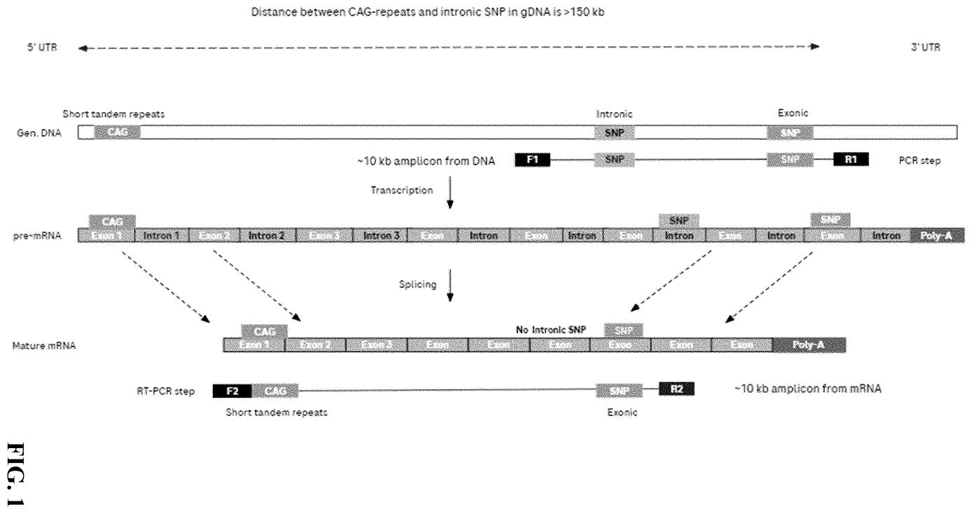

FIG. 1 indicates the distant separation between tandem-repeat region (i.e. CAG) and intronic or exonic SNP of interest. Distance is being represented on genomic DNA level and may span up to 200 kb nucleotides. As the target intronic SNP is far away from the CAG repeat (>150 kb), the locus including both cannot be PCR amplified using genomic DNA as a template. However, the intronic target SNP and exonic reference SNP are separated from each other by only around 10 kb and the locus can be amplified with a PCR assay (i.e., using a set of oligonucleotide primers F1 and R1) using genomic DNA as a template. The transcription and mRNA maturation of the gene target is characterized by a splicing event that leads into reduction of the distance between CAG tandem repeats and exonic reference SNP, which allows for reverse transcription and PCR of a 10 kb amplicon from a single molecule (i.e., using a set of oligonucleotide primers F2 and R2) by using mRNA as a starting material.

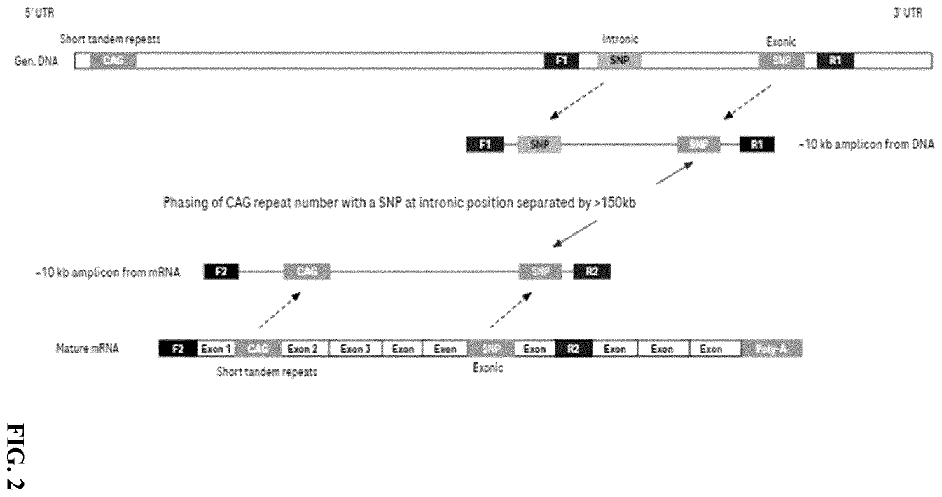

FIG. 2 depicts the resulting two amplicons, a first amplicon generated from a genomic DNA and containing an intronic target SNP and an exonic reference SNP and a second amplicon generated from cDNA derived from mature mRNA and containing CAG tandem repeats and an exonic reference SNP. Herein, the exonic reference SNP in both amplicons is the same. Thereby, the hybrid analysis allows for phasing of at least one distant intronic SNP with a number of CAG tandem repeats by an alignment of the exonic reference SNP between two amplicons. In some aspects, more than one exonic reference SNP may be used to further improve phasing accuracy.

### II. Definitions

A “companion diagnostic” is a diagnostic test used as a companion to a therapeutic drug or treatment to determine its applicability to specific patients or specific patient groups and to thereby stratify patients according to a certain genomic profile. Companion diagnostics refers to diagnostic tests that are developed alongside a specific drug or therapy (e.g. antibody, small molecule or antisense oligonucleotide) to select or exclude patients or groups of patients for treatment with that particular drug based on their biological characteristics that determine responders and non-responders to the therapy. These tests often involve the identification of specific genetic biomarkers or mutations that are associated with the disease or condition being treated or that prospectively help to predict likely response or severe toxicity.

The term “biomarker” can refer to any detectable marker used to differentiate individual samples, e.g., cancer versus non-cancer samples. Biomarkers include modifications (e.g., methylation of DNA, phosphorylation of protein), differential expression, and mutations or variants (e.g., single nucleotide variations, insertions, deletions, splice variants, and fusion variants). A biomarker can be detected in a DNA, RNA, and/or protein sample.

The terms “nucleic acid,” “polynucleotide,” and “oligonucleotide” refer to polymers of nucleotides (e.g., ribonucleotides or deoxyribonucleotides) and includes naturally-occurring (e.g., adenosine, guanidine, cytosine, uracil and thymidine), and non-naturally occurring (human-modified) nucleic acids. The term is not limited by length (e.g., number of monomers) of the polymer. Nucleoside triphosphates that contain ribose as the sugar are conventionally abbreviated as NTPs, while nucleoside triphosphates containing deoxyribose as the sugar are abbreviated as dNTPs. “Nucleic acid” shall mean, unless otherwise specified, any nucleic acid molecule, including, without limitation, DNA, RNA and hybrids thereof. In an embodiment the nucleic acid bases that form nucleic acid molecules can be the bases A, C, G, T and U, as well as derivatives thereof (A—Adenine; C—Cytosine; G—Guanine; T—Thymine; U—Uracil). “Derivatives” or “analogues” of these bases are well known in the art, and are exemplified in PCR Systems, Reagents and Consumables (Perkin Elmer Catalog 1996-1997, Roche Molecular Systems, Inc., Branchburg, New Jersey, USA). A nucleic acid may be single-stranded or double-stranded and will generally contain 5′-3′ phosphodiester bonds, although in some cases, nucleotide analogs may have other linkages. Monomers are typically referred to as nucleotides. The term “non-natural nucleotide” or “modified nucleotide” refers to a nucleotide that contains a modified nitrogenous base, sugar or phosphate group, or that incorporates a non-natural moiety in its structure. Examples of non-natural nucleotides include LNA, dideoxynucleotides, biotinylated, aminated, deaminated, alkylated, benzylated and fluorophor-labeled nucleotides. “LNA” refers to Locked Nucleic Acid. LNA is a modified RNA nucleotide in which the ribose moiety is modified with an extra bridge connecting the 2′ oxygen and 4′ carbon. LNA nucleotides can be mixed with DNA or RNA residues in any position in an oligonucleotide and hybridize with DNA or RNA according to Watson-Crick base-pairing rules. The locked ribose conformation enhances hybridization properties (e.g., increases melting temperatures).

A “nucleotide residue” is a single nucleotide in the state it exists after being incorporated into, and thereby becoming a monomer of, a polynucleotide. Thus, a nucleotide residue is a nucleotide monomer of a polynucleotide, e.g. DNA, which is bound to an adjacent nucleotide monomer of the polynucleotide through a phosphodiester bond at the 3′ position of its sugar and is bound to a second adjacent nucleotide monomer through its phosphate group, with the exceptions that (i) a 3′ terminal nucleotide residue is only bound to one adjacent nucleotide monomer of the polynucleotide by a phosphodiester bond from its phosphate group, and (ii) a 5′ terminal nucleotide residue is only bound to one adjacent nucleotide monomer of the polynucleotide by a phosphodiester bond from the 3′ position of its sugar.

Because of well-understood base-pairing rules, determining the identity (of the base) of dNTP analogue (or rNTP analogue) incorporated into a primer or DNA extension product (or RNA extension product) by measuring the unique electrical signal of the tag translocating through the nanopore, and thereby the identity of the dNTP analogue (or rNPP analogue) that was incorporated, permits identification of the complementary nucleotide residue in the single stranded polynucleotide that the primer or DNA extension product (or RNA extension product) is hybridized to. Thus, if the dNPP analogue that was incorporated comprises an adenine, a thymine, a cytosine, or a guanine, then the complementary nucleotide residue in the single stranded DNA is identified as a thymine, an adenine, a guanine or a cytosine, respectively. The purine adenine (A) pairs with the pyrimidine thymine (T). The pyrimidine cytosine (C) pairs with the purine guanine (G). Similarly, with regard to RNA, if the rNPP analogue that was incorporated comprises an adenine, a uracil, a cytosine, or a guanine, then the complementary nucleotide residue in the single stranded RNA is identified as a uracil, an adenine, a guanine or a cytosine, respectively.

The terms “cell-free nucleic acids”, “cell-free DNA”, “cell-free RNA” and like terms in the context of the present disclosure refers to a non-tissue sample (e.g., liquid biopsy) from an individual that has been processed to largely remove cells. Examples of non-tissue samples include blood and blood components, urine, saliva, tears, mucus, etc.

A “precursor mRNA” or pre-mRNA is a primary molecule in the eukaryotic transcription process, produced from a DNA template inside the cell nuclei. The pre-mRNA molecule has both: coding (exons) and non-coding (introns) sequences and is being subjected to a maturation including splicing step where intronic regions are removed and molecule becomes mRNA after processing.

“Mature mRNA” is a eukaryotic RNA transcript that has been spliced and processed and is ready for translation in the course of protein synthesis. Unlike the eukaryotic RNA immediately after transcription known as precursor mRNA, mature mRNA consists exclusively of exons and has all introns removed.

A “single-nucleotide polymorphism” (SNP) is a germline substitution of a single nucleotide at a specific position in the genome and is present in a sufficiently large fraction of the population (1% or more). The genomic distribution of SNPs is not homogenous; SNPs occur in non-coding regions (intronic regions) more frequently than in coding regions (exonic regions). As used herein, a “distant single nucleotide polymorphism” is located at a distance of more than 10 kb on the same nucleic acid relative to another single nucleotide polymorphism or a short tandem repeat. In some instances, the distance may be more than 20 kb, 30 kb, 40 kb, 50 kb, 60 kb, 70 kb, 80 kb, 90 kb, 100 kb, 110 kb, 120 kb, 130 kb, 140 kb, 150 kb, 160 kb 170 kb, 180 kb, or 190 kb. In some instances, the distance may be between 10-200 kb, 10-160 kb, 20-200 kb, 20-160 kb, 50-200 kb, 50-160 kb, 100-200 kb, 100-160 kb or 150-200 kb.

An “exonic reference single nucleotide polymorphism” or “exonic reference SNP” as used herein enables long distance phasing as it can be covered by a cDNA amplicon (reverse transcribed from mature mRNA), which does not include introns, as well as by a DNA (e.g., a genomic DNA) amplicon. Thus, the “exonic reference SNP” also allows for the phasing and analysis of a heterozygous and a homozygous exonic or intronic target SNP, while certain scenarios require the “exonic reference SNP” being heterozygous (e.g., when the target SNP is heterozygous). In particular, an optimal “exonic reference SNP” should be polymorphic and heterozygous if the distant target single nucleotide polymorphism (SNP) of interest (intronic or exonic) is heterozygous in a given individual. Thereby, the “exonic reference SNP” may be used to align nucleic acid sequences of a first amplification product derived from a target DNA nucleic acid (e.g., a genomic DNA) and a second amplification product derived from a target cDNA (reverse transcribed from mature mRNA) nucleic acid based on the position of the “exonic reference SNP”.

The term “short tandem repeat” (STR) may also be referred to as “microsatellite” and represent a tract of repetitive DNA in which certain DNA motifs (ranging in length from one to six or more base pairs) are repeated, typically 5-50 times. Microsatellites occur at thousands of locations within an organism's genome making up around 3% of the human genome and are often found in introns and intergenic regions, but also occur in exons. They have a higher mutation rate than other areas of DNA leading to high genetic diversity. STRs may be involved in the onset of disorders, such as, e.g., Huntington's disease. Herein, the abnormal expansion of tandem repeats consisting of a sequence of cytosine-adenine-guanine (CAG) nucleotides in the huntingtin gene chromosome 4, exon 1 are thought to be causative for the onset of the disease. While a normal huntingtin gene typically has 10-35 CAG repeats, individuals with Huntington's disease exhibit a number of CAG repeats ranging from 36 to over 100.

The term “phasing” refers to assigning genetic variants to their homologous chromosome of origin. Humans have two copies of every chromosome, one inherited maternally and other paternally. In the context of phasing the STR and SNP, the aim is to identify which SNP allele is on the same chromosome (i.e. in the haploid genome) with a specific length of STR. In the context of phasing two or more SNPs, the aim is to identify which SNP alleles of the first and second (and third . . . ) SNP are on the same chromosome.

The term “primer” refers to a short nucleic acid (an oligonucleotide) that acts as a point of initiation of polynucleotide strand synthesis by a nucleic acid polymerase under suitable conditions. Polynucleotide synthesis and amplification reactions typically include an appropriate buffer, dNTPs and/or rNTPs, and one or more optional cofactors, and are carried out at a suitable temperature. A primer typically includes at least one target-hybridized region that is at least substantially complementary to the target sequence (e.g., having 0, 1, or 2 mismatches). For the purposes of the present disclosure, this region is typically about 4 to about 10 nucleotides in length, e.g., 5-8 nucleotides. A “primer pair” refers to a forward and reverse primer that are oriented in opposite directions relative to the target sequence, and that produce an amplification product in amplification conditions. The terms “forward” and “reverse” are assigned arbitrarily. One of ordinary skill in the art will understand that forward and reverse primers (primer pair) define the borders of an amplification product. In some embodiments, multiple primer pairs rely on a single common forward or reverse primer. For example, multiple allele-specific forward primers can be considered part of a primer pair with the same, common reverse primer, e.g., if the multiple alleles are in close proximity to each other. A “set of primers” or “primer set” can refer to a primer pair, or more than one primer pair designed to work together in a single multiplex reaction.

As used herein, “probe” means any molecule that is capable of selectively binding to a specifically intended target biomolecule, for example, a nucleic acid sequence of interest that hybridizes to the probes. The probe is detectably labeled with at least one non-nucleotide moiety. In some embodiments, the probe is labeled with a fluorophore and quencher.

The words “complementary” or “complementarity” refer to the ability of a nucleic acid in a polynucleotide to form a base pair with another nucleic acid in a second polynucleotide. For example, the sequence A-G-T (A-G-U for RNA) is complementary to the sequence T-C-A (U-C-A for RNA). Complementarity may be partial, in which only some of the nucleic acids match according to base pairing, or complete, where all the nucleic acids match according to base pairing. A probe or primer is considered “specific for” a target sequence if it is at least partially complementary to the target sequence. Depending on the conditions, the degree of complementarity to the target sequence is typically higher for a shorter nucleic acid such as a primer (e.g., greater than 80%, 90%, 95%, or 98%) than for a longer sequence. In some embodiments, primers and/or probes are 100% complementary to the targeted sequence.

The term “specifically amplifies” indicates that a primer set amplifies a target sequence more than non-target sequence at a statistically significant level. The term “specifically detects” indicates that a probe will detect a target sequence more than non-target sequence at a statistically significant level. As will be understood in the art, specific amplification and detection can be determined using a negative control, e.g., a sample that includes the same nucleic acids as the test sample, but not the target sequence or a sample lacking nucleic acids. For example, primers and probes that specifically amplify and detect a target sequence result in a Ct that is readily distinguishable from background (non-target sequence), e.g., a Ct that is at least 2, 3, 4, 5, 5-10, 10-20, or 10-30 cycles less than background. The term “allele-specific” PCR refers to amplification of a target sequence using primers that specifically amplify a particular allelic variant of the target sequence. Typically, the forward or reverse primer includes the exact complement of the allelic variant at that position.

The terms “identical” or “percent identity,” in the context of two or more nucleic acids, or two or more polypeptides, refer to two or more sequences or subsequences that are the same or have a specified percentage of nucleotides, or amino acids, that are the same (e.g., about 60% identity, e.g., at least any of 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” Percent identity is typically determined over optimally aligned sequences, so that the definition applies to sequences that have deletions and/or additions, as well as those that have substitutions. The algorithms commonly used in the art account for gaps and the like. Typically, identity exists over a region comprising a sequence that is at least about 8-25 amino acids or nucleotides in length, or over a region that is 50-100 amino acids or nucleotides in length, or over the entire length of the reference sequence.

The terms “isolate,” “separate,” “purify,” and like terms are not intended to be absolute. For example, isolation of DNA or genomic DNA does not require 100% of non-DNA molecules to be removed. One of skill in the art will recognize an acceptable level of purity for a given situation.

The term “amplification product” refers to the product of an amplification reaction. The amplification product includes the primers used to initiate each round of polynucleotide synthesis. An “amplicon” is the sequence targeted for amplification, and the term can also be used to refer to amplification product. The 5′ and 3′ borders of the amplicon are defined by the forward and reverse primers. A “reverse transcription product,” “RT product,” and like terms refers to a cDNA molecule produced by elongation of an RT primer on an RNA template by a polymerase with reverse transcriptase activity.

The term “kit” refers to any manufacture (e.g., a package or a container) including at least one reagent, such as a nucleic acid probe or probe pool or the like, for specifically amplifying, capturing, tagging/converting or detecting RNA or DNA as described herein.

The term “amplification conditions” refers to conditions in a nucleic acid amplification reaction (e.g., PCR amplification) that allow for hybridization and template-dependent extension of the primers. The term “amplicon” or “amplification product” refers to a nucleic acid molecule that contains all or a fragment of the target nucleic acid sequence and that is formed as the product of in vitro amplification by any suitable amplification method. The term “generate an amplification product” when applied to primers, indicates that the primers, under appropriate conditions (e.g., in the presence of a nucleotide polymerase and NTPs), will produce the defined amplification product. Various PCR conditions are described in PCR Strategies (Innis et al., 1995, Academic Press, San Diego, CA) at Chapter 14; PCR Protocols: A Guide to Methods and Applications (Innis et al., Academic Press, NY, 1990).

“Nanopore” is defined as a structure that has a nanoscale channel that can pass ions in solution from one side to the other. Examples of nanopores are protein nanopores (e.g., (α-hemolysin and other multi-subunit porins), synthetic nanopores, and hybrid protein/synthetic nanopores. In relevant embodiments, these nanopores are inserted into a natural or artificial membrane that would otherwise serve to prevent passage of ions and other molecules. The width of the nanopore channel should allow polymers such as single stranded DNA to pass through, typically upon application of a voltage gradient across the membrane. During their transit, they will reduce the ionic current at a given voltage, due to their size, charge or other characteristics. “Nanopore” includes, for example, a structure comprising (a) a first and a second compartment separated by a physical barrier, which barrier has at least one pore with a diameter, for example, of from about 1 to 10 nm, and (b) a means for applying an electric field across the barrier so that a charged molecule such as DNA, nucleotide, nucleotide analogue, or tag, can pass from the first compartment through the pore to the second compartment. The nanopore ideally further comprises a means for measuring the electronic signature of a molecule passing through its barrier. The nanopore barrier may be synthetic or naturally occurring in part. Barriers can include, for example, lipid bilayers having therein α-hemolysin, oligomeric protein channels such as porins, and synthetic peptides and the like. Barriers can also include inorganic plates having one or more holes of a suitable size. Herein “nanopore”, “nanopore barrier” and the “pore” in the nanopore barrier are sometimes used equivalently.

Nanopore devices are known in the art and nanopores and methods employing them are disclosed in U.S. Pat. Nos. 7,005,264; 7,846,738; 6,617,113; 6,746,594; 6,673,615; 6,627,067; 6,464,842; 6,362,002; 6,267,872; 6,015,714; 5,795,782; and U.S. Publication Nos. 2004/0121525, 2003/0104428, and 2003/0104428, each of which are hereby incorporated by reference in their entirety.

“Nanopore arrays” are chips containing many individual nanopores at known positions; each nanopore can be separately interrogated electronically (allowing single molecule electronic nanopore-based sequencing by synthesis).

“Nanopore-detectable tag” (also referred to as a “nanopore tag”) is a molecule, usually a polymer, covalently attached to the nucleotides in a Nanopore SBS reaction. A different nanopore tag is typically attached to each nucleotide, A, C, G and T (or U), so as to elicit different ionic current blockade signals as they pass through the channel of the nanopore, when a voltage gradient is applied across the membrane.

Nanopore sequencing by synthesis (also referred to as “Nanopore SBS”) refers to the approach described previously by us (Kumar et al. 2012; Fuller et al. 2016; Stranges et al. 2016) in which tags that are attached to nucleotides can be distinguished by their effect on ionic currents passing through nanopores as these modified nucleotides are added to a growing DNA strand. Measurements can be made while tagged nucleotides are still part of the ternary complex, or after their tags are released by the polymerase reaction.

The terms “individual”, “subject”, and “patient” are used interchangeably herein. The individual can be pre-diagnosis, post-diagnosis but pre-therapy, undergoing therapy, or post-therapy. In the context of the present disclosure, the individual is typically seeking medical care.

The term “sample” or “biological sample” refers to any composition containing or presumed to contain nucleic acid. The term includes purified or separated components of cells, tissues, or blood, e.g., DNA, RNA, proteins, cell-free portions, or cell lysates. The sample can be FFPET, e.g., from a tumor or metastatic lesion. The sample can also be from frozen or fresh tissue, or from a liquid sample, e.g., blood or a blood component (plasma or serum), urine, semen, saliva, sputum, mucus, semen, tear, lymph, cerebral spinal fluid, mouth/throat rinse, bronchial alveolar lavage, material washed from a swab, etc. Samples also may include constituents and components of in vitro cultures of cells obtained from an individual, including cell lines. The sample can also be partially processed from a sample directly obtained from an individual, e.g., cell lysate or blood depleted of red blood cells. A tumor sample can include tissue from a tumor, or a sample that includes DNA from a tumor, e.g., ctDNA in the blood of a cancer patient.

The term “obtaining a sample from an individual” means that a biological sample from the individual is provided for testing. The obtaining can be directly from the individual, or from a third party that directly obtained the sample from the individual. The sample could be taken before treatment, during treatment or post-treatment. The sample may be taken from a patient who is suspected of having, or is diagnosed as having disease X, and hence is likely in need of treatment or from a normal individual who is not suspected of having any disorder. Treatment regimen, (higher/lower/more frequent/less frequent) dose.

The term “assessing disease X” is used to indicate that the methods disclosed herein will aid a medical professional including, e.g., a physician to assess whether an individual has disease X or is at risk of developing disease X or prognosing the course of disease X. The presence of biomarker Y, a combination of biomarkers Y;Z; . . . or a ratio of biomarkers Y;Z; . . . in the sample indicates that the individual has disease X or that the individual is at risk of developing disease X or prognosing the course of disease X. In one embodiment the term assessing disease X is used to indicate that the method according to the present invention will aid the medical professional to assess whether an individual has disease X or not. In this embodiment the presence of biomarker Y in the sample indicates that the individual has disease X, i.e., the presence of biomarker Y is indicative of the presence of disease X in the individual at/above/below the reference level. In certain embodiments, the term “at the reference level” refers to a level of the biomarker in the sample from the individual or patient that is essentially identical to the reference level or to a level that differs from the reference level by up to 1%, up to 2%, up to 3%, up to 4%, up to 5%.

The term “providing therapy for an individual” means that the therapy is prescribed, recommended, or made available to the individual. The therapy may be actually administered to the individual by a third party (e.g., an in-patient injection), or by the individual herself. The phrase “selecting a therapy” as used herein refers to using the information or data generated relating to the level or presence of biomarker Y in a sample of a patient to identify or selecting a therapy for a patient. In some embodiment, the therapy may comprise drug D. In some embodiments, the phrase “identifying/selecting a therapy” includes the identification of a patient who requires adaptation of an effective amount of drug D being administered. In some embodiments, recommending a treatment includes recommending that the amount of drug D being administered is adapted. The phrase “recommending a treatment” as used herein also may refer to using the information or data generated for proposing or selecting a therapy comprising drug D for a patient identified or selected as more or less likely to respond to the therapy comprising drug D. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the level of biomarker Y to a reference level. In some embodiments, the information or data includes an indication that biomarker Y is present or absent in the sample. In some embodiments, the information or data includes an indication that a therapy comprising drug D is suitable for the patient.

The terms “label,” “tag,” “detectable moiety,” and like terms refer to a composition detectable by spectroscopic, photochemical, biochemical, immunochemical, chemical, or other physical means. For example, useful labels include fluorescent dyes (fluorophores), luminescent agents, radioisotopes (e.g., ³²P, ³H), electron-dense reagents, or an affinity-based moiety, e.g., a poly-A (interacts with poly-T) or poly-T tag (interacts with poly-A), a His tag (interacts with Ni), or a streptavidin tag (separable with biotin). One of skill will understand that a detectable label conjugated to a nucleic acid is not naturally occurring.

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Springs Harbor Press (Cold Springs Harbor, N.Y. 1989). The term “a” or “an” is intended to mean “one or more.” The terms “comprise,” “comprises,” and “comprising,” when preceding the recitation of a step or an element, are intended to mean that the addition of further steps or elements is optional and not excluded.

### III. Nucleic Acid Samples

Samples for biomarker detection can be obtained from any source suspected of containing substantial amounts of non-fragmented nucleic acids or large fragments (>10 kb) of nucleic acids, e.g., tissue (including tumor tissue), blood (including cell-free nucleic acids such as cell-free DNA and cell-free RNA), skin, swab (e.g., buccal, vaginal), urine, saliva, etc. Methods for isolating nucleic acids from biological samples are known, e.g., as described in Sambrook, and several kits are commercially available (e.g., High Pure RNA Isolation Kit, High Pure Viral Nucleic Acid Kit, and MagNA Pure LC Total Nucleic Acid Isolation Kit, DNA Isolation Kit for Cells and Tissues, DNA Isolation Kit for Mammalian Blood, High Pure FFPET DNA Isolation Kit, available from Roche). In the context of the presently disclosed methods, genomic DNA and RNA can be collected and isolated.

### IV. Detailed Description of the Workflows

In the following a detailed description of an exemplary laboratory workflow is provided using sample preparation, amplification, library preparation and sequencing analysis and an exemplary subsequent bioinformatics workflow including the analysis of the sequencing data exemplifying the implementation of the disclosed methods.

### Laboratory Workflow

Sample of interest (e.g., blood or tissue) is processed with a DNA and RNA co-extraction method (e.g., AllPrep DNA/RNA Micro Kit, Qiagen) according to the manufacturer's protocol. Alternatively, the sample of interest may be split up in two portions, while DNA may be extracted from a first portion (e.g., using DNeasy Blood & Tissue Kit, Qiagen) and RNA may be extracted from a second portion of the sample (e.g., using RNeasy Kit, Qiagen) according to the manufacturer's protocol. The isolated nucleic acid material is analyzed with a quality control workflow (e.g., NanoDrop™ spectrophotometer, Qubit™ fluorometer) according to the manufacturer's protocol and subsequently subjected to an amplification reaction based on polymerase chain reaction (PCR) or an amplification reaction based on reverse transcription and polymerase chain reaction (RT-PCR). Aforementioned reactions (i.e., PCR and RT-PCR) are performed on genomic DNA and mRNA accordingly and utilize standard reagents for a long-amplicon generation (i.e., polymerase, reaction buffer, primers etc.) and standard equipment (e.g., pipettes, tips, laboratory tubes, PCR-hood and PCR thermocycler). Designs for custom primers for performing PCR and RT-PCR amplification reactions to generate DNA and cDNA amplicons can be obtained using the NCBI Primer Blast or Primer3 open-source algorithms (National Library of Medicine, Bethesda, MD, USA). PCR and RT-PCR reactions are executed according to manufacturer's protocols (e.g., Expand™ High Fidelity PCR System, Roche). Herein, the amount of PCR and RT-PCR cycles is dependent on the amount and quality of the isolated nucleic acids as starting material. DNA and cDNA amplicons of interest are subjected to a clean-up step according to standard methodologies and manufacturer's documentation (e.g., Agencourt AMPure XP magnetic beads, Beckman Coulter; or Blue Pippin Prep, Sage Science) to remove excess of unused amplification primers or short off-target molecules. Purified amplicons are tested with a quality control workflow (e.g. NanoDrop™ spectrophotometer, Qubit™ fluorometer or Bioanalyzer, Agilent Technologies, Santa Clara, CA, USA) according to manufacturer's protocols and recommendations. Two amplicons (i.e., DNA and cDNA) from a single sample are pooled in equimolar concentration and ligated with a barcode index containing sequencing adapters (e.g., hairpin loop for PacBio sequencing using a library preparation kit, Pacific Biosciences of California, Inc., Menlo Park, USA) according to the manufacturer's protocol and further combined into a final sequencing library pool. Such a generated sample is quantified with a Qubit™ spectrophotometer (e.g., Broad Range DNA Kit, Thermo Fisher Scientific) and loaded onto the sequencing device (e.g., Sequel system, Pacific Biosciences of California, Inc., Menlo Park, USA) and processed according to the manufacturer's device instructions.

### Bioinformatics Workflow

The raw data generated with a one of the long-read sequencing devices (e.g., Sequel system, Pacific Biosciences of California, Inc., Menlo Park, USA; or GridION system, Oxford Nanopore Technologies Ltd., Oxford, UK) may be bioinformatically processed according to the flow chart as shown in FIG. 8. Herein, in a first step the sequencing raw data (100) can be basecalled with an onboard software into FASTQ reads (110). Such generated data can be transferred onto a local Linux based server for a bioinformatic data analysis. Available reads are demultiplexed or consensus called (120) with open source algorithms (e.g. LIMA or pbCCS) and aligned against a whole human genome reference (e.g. BWA, STAR or minimap2) according to presence or absence of introns (130). Outcome of the alignment allows for a single molecule SNP calling (MUSCLE, SNVer, VarScan, Samtools/mpileup) and extraction of exonic or intronic statistical values (131). While the number of short tandem repeats from a cDNA reads can be quantified with a Tandem Repeat Genotyper, RepeatAnalysisTools or DeepRepeat software (132). DNA and cDNA data undergo similarity clustering (e.g. UCLUST, VSEARCH or pbAA), allowing better resolution of CAG tandem repeats or exonic/intronic SNP. Information from a DNA and cDNA data are combined into a single CSV file and binned according to the amount of CAG tandem repeats or exonic/intronic SNP. Resulting bins for homo- and heterozygotes can be visualized with an Integrative Genomics Viewer (IGV) and plotted in tables for each separate step to finally indicate which target SNP allele is in the same phase as the mutant CAG repeat (140). Statistical analysis may be performed to remove noisy signal or chimera reads and increase the final statistical accuracy.

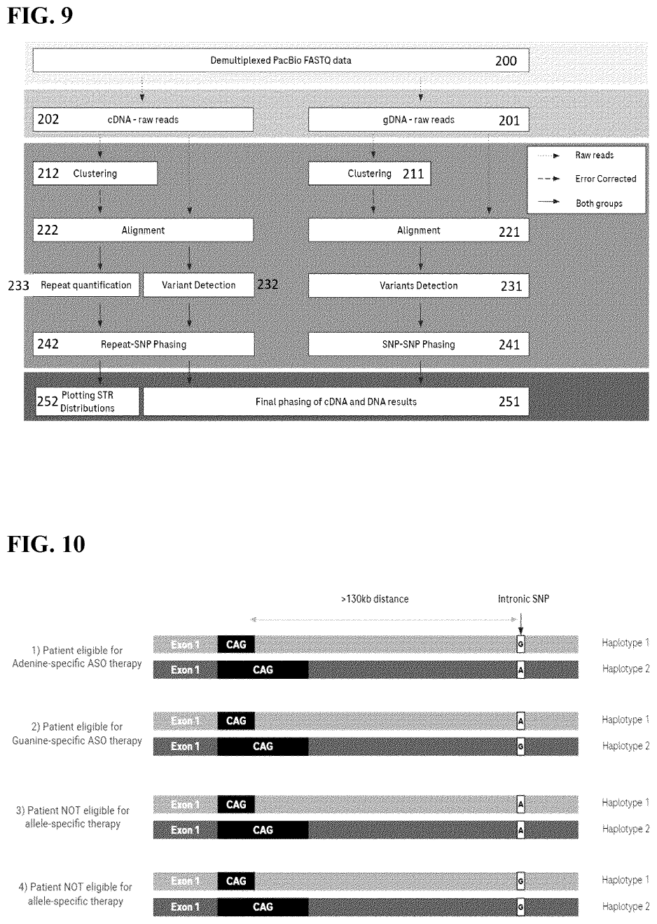

In a related workflow, the raw data generated with one of the long-read sequencing devices (e.g., Sequel system, Pacific Biosciences of California, Inc., Menlo Park, USA) may be bioinformatically processed according to the flow chart as shown in FIG. 9. The method uses multiple steps of reads filtration to generate the highest quality data for clustering and alignment. Statistical analysis is performed with validated open source programs and combines the signal into a final phasing report and STR distributions. In the first step, the raw sequencing data is transferred via local area network (LAN) onto a server for a post-processing and statistical data analysis. The consensus calling is executed with a pbCCS software into FASTQ reads, allowing filtering out shorter amplicons and reads with lower consensus quality or incomplete sequencing passes. Generated consensus reads are demultiplexed (200) with an open source algorithm (i.e. LIMA) and data is saved separately for cDNA amplicons (202) and gDNA amplicons (201). Subsequently, high quality reads are clustered (212, 211) with a PacBio Amplicon Analysis (pbAA). The raw and clustered bins are aligned (222, 221) against a whole human genome reference according to presence or absence of introns. Binning of reads is performed to remove noisy signals (e.g., PCR chimera) and increase the final statistical accuracy. High quality data is used for extraction of gene positions, for cDNA (232) including exonic SNP and motifs of interest (e.g., CAG, CAA, CCG, CCA, CGG) and for gDNA (231, 232) including exonic and intronic SNP coordinates (233). Outcome of the alignment allows for a single molecule SNP calling and subsequent extraction of exonic and intronic statistical metrics for target positions (241). The number of short tandem repeats from a cDNA reads is quantified with a RepeatAnalysisTools (233). gDNA and cDNA reads subjected to similarity clustering (i.e. pbAA), allow for better resolution of STRs and exonic/intronic SNPs (242), while unclustered reads allow for better quantitative plotting of reads per haplotype. Information from both aforementioned workflows (i.e. error corrected and uncorrected gDNA/cDNA) are combined into a single file and binned according to the amount of STRs or exonic/intronic SNP. Resulting bins for homo- and heterozygotes are visualized with waterfall images and represent STRs (252). Primary outcome of the overall analysis indicates which intronic SNP allele is in phase with the mutant or wild type STRs (251).

### Patient Stratification Workflow

Generated results may be analyzed to identify the presence of desired intronic SNP allele being in phase with mutant STR allele (e.g., a CAG repeat). FIG. 10 visualizes the final four possible phasing outcomes (i.e., haplotype consisting of the CAG repeat and the intronic target SNP). It is to be noted that only combinations with heterozygous target SNP allow for allele-specific treatment using antisense oligonucleotide (ASO) based therapies. As shown in FIG. 10 for an Adenine-specific ASO the following haplotypes would be eligible:
1) Haplotype 1: mutant STR+Adenine SNP and
   Haplotype 2: wild type STR+Guanine SNP

The inverse SNP allele:
2) Haplotype 1: mutant STR+Guanine SNP and
   Haplotype 2: wild type STR+Adenine SNP
      would cause depletion of wild type STR mRNA and preservation of mutant STR mRNA. Thus, treatment of a patient with the inverse SNP allele 2) would require the use of a Guanine-specific ASO. Remaining homozygous combinations 3 (homozygous SNP A/A) or 4 (homozygous SNP G/G) would cause depletion of wild type and mutant STR mRNA or would result in no on-target lowering of either of the STR alleles after ASO administration.

### V. Kits

Provided herein are kits for determining the nucleic acid sequence of at least one distant single nucleotide polymorphism of interest and short tandem repeats within the same target gene locus of nucleic acids isolated from a biological sample is provided, the kit comprising a first set of oligonucleotide primers to produce a first amplification product comprising the at least one single nucleotide polymorphism of interest and at least one exonic reference single nucleotide polymorphism in the same gene locus; and a second set of oligonucleotide primers to produce a second amplification product comprising the at least one single nucleotide polymorphism of interest and said at least one exonic reference single nucleotide polymorphism. Herein, the kit is adapted for performing any of the methods disclosed herein. In a particular embodiment, the first set of oligonucleotide primers comprises an oligonucleotide primer comprising the nucleic acid sequence of SEQ ID NO:1 and an oligonucleotide primer comprising the nucleic acid sequence of SEQ ID NO:2. In another particular embodiment, the second set of oligonucleotide primers comprises an oligonucleotide primer comprising the nucleic acid sequence of SEQ ID NO:3, an oligonucleotide primer comprising the nucleic acid sequence of SEQ ID NO:4 and an oligonucleotide primer comprising the nucleic acid sequence of SEQ ID NO:5. In some embodiments, the kit comprises a sample collection vessel (e.g., tube, vial, multi-well plate or multi-vessel cartridge).

In some embodiments, the kit includes reagents and/or components for nucleic acid purification. For example, the kit can include a lysis buffer (e.g., comprising detergent, chaotropic agents, buffering agents, etc.), enzymes or reagents for denaturing proteins or other undesired materials in the sample (e.g., proteinase K), enzymes to preserve nucleic acids (e.g., DNase and/or RNase inhibitors). In some embodiments, the kit includes components for nucleic acid separation, e.g., solid or semi-solid matrices such as chromatography matrix, magnetic beads, magnetic glass beads, glass fibers, silica filters, etc. In some embodiments, the kit includes wash and/or elution buffers for purification and release of nucleic acids from the solid or semi-solid matrix. For example, the kit can include components from MagNA Pure LC Total Nucleic Acid Isolation Kit, DNA Isolation Kit for Mammalian Blood, High Pure or MagNA Pure RNA Isolation Kits (Roche), DNeasy or RNeasy Kits (Qiagen), PureLink DNA or RNA Isolation Kits (Thermo Fisher), etc.

The kit can further include reagents for amplification, e.g., reverse transcriptase, DNA polymerase, dNTPs, buffers, and/or other elements (e.g., cofactors or aptamers) appropriate for reverse transcription and/or amplification. Typically, the reagent mixture(s) is concentrated, so that an aliquot is added to the final reaction volume, along with sample (e.g., RNA or DNA), enzymes, and/or water. In some embodiments, the kit further comprises reverse transcriptase (or an enzyme with reverse transcriptase activity), and/or DNA polymerase (e.g., thermostable DNA polymerase such as Taq, ZO5, and derivatives thereof).

In some embodiments, the kit further includes consumables, e.g., plates or tubes for nucleic acid preparation, tubes for sample collection, or plates, tubes, or microchips for PCR or qRT-PCR. In some embodiments, the kit further includes instructions for use, reference to a website, or software, e.g., for further processing of sequencing data.

### EXAMPLES

### Example 1

Amplification of Genomic DNA and Reverse Transcription of mRNA from HTT Gene

Huntington's disease is a rare autosomal dominant genetic disorder, which is caused by an abnormal expansion of tandem repeats consisting of a sequence of cytosine-adenine-guanine (CAG) nucleotides in the huntingtin gene (HTT) chromosome 4, exon 1. A normal HTT typically has 10-35 CAG repeats, but in individuals with Huntington's disease, the number of CAG repeats can range from 36 to over 100. In order to treat the disease, ideally only the mutant HTT would be suppressed, leaving the wild-type HTT intact. Targeting heterozygous SNPs in proximity of the CAG repeat would allow for such an allele-specific approach. Several SNPs with high allele-frequency, including the SNP rs7685686 in intron 42, have been identified in the HTT gene as a candidate for targeted treatment. However, an assay to identify heterozygous patients that have the specific SNP allele in phase with the mutant CAG repeat, is a prerequisite for such a selective treatment. Phasing of the CAG repeats in Exon 1 and the SNP in Intron 42 of HTT is not possible using standard methodologies due to the large distance of more than 130 kb between these genetic variants. Moreover, only using RNA as a starting material to reduce the distance between the CAG repeat and distant SNPs, is not possible for intronic SNPs. Thus, a novel laboratory workflow, utilizing both genomic DNA and RNA, was developed to enable the phasing analysis of STRs and distant, even intronic SNPs within the same gene locus.

Thirteen samples from NIGMS Human Genetic Cell Repository at the Coriell Institute for Medical Research were processed as described below. Genomic DNA and total RNA were isolated using the co-extraction method (AllPrep DNA/RNA Micro Kit, Qiagen) according to the manufacturer's protocol. Extracted nucleic acid was processed with quality control workflow based on quantification of concentration and purity with use of Qubit HS DNA/RNA kit and nanodrop spectrophotometer accordingly. Samples characterized by a high concentration and correct 260/230, 260/280 quality and RNA Integrity Number (RIN) were subjected for PCR and RT-PCR.

The DNA assay of ~10 kb amplicons, required input of minimum 100 ng of high quality material and was mixed with 10 ul of 5× PrimeSTAR GXL Buffer, 4 uL of dNTPS Mixture (200 uM each), 0.7 uL of 15 uM forward and reverse primer, 1 uL of PrimerSTAR GXL DNA Polymerase and 13.6 uL of nuclease-free water (i.e. 30 uL of Master Mix), while the input genomic DNA was added at 5 ng/uL and total 20 uL volume (50 uL final reaction volume). The amplification reaction was carried out in a preheated thermocycler with following conditions: pre-incubation at 98° C. for 1 min followed by 30× cycles at 98° C. for 20 sec, 58° C. for 15 sec, followed by incubation at 68° C. for 15 min. The reaction was stopped by lowering the temperature down to 4° C. for infinite time. Amplicons were purified with AMPure® PB Beads (100-265-900, Pacific Biosciences) according to manufacturer protocol at 0.45× ratio. Quantity of the amplicons were measured with Qubit BR DNA assay while size of them was verified with TapeStation, Genomic DNA Screen Tapes. Generated amplicons were subjected for a second purification with AMPure® PB Beads (100-265-900, Pacific Biosciences) according to manufacturer protocol at 3.1× ratio to remove potential <7 kb fragments still present After the second purification, quantity of the amplicons were measured again with Qubit BR DNA assay while size of them was verified with TapeStation, Genomic DNA Screen Tapes.

The cDNA assay of ~10 kb amplicons required a minimum of 1000 ng of high integrity total RNA for the primary step. The reaction comprises gDNA removal by mixing 1 uL of 10× exDNase buffer with 1 uL of exDNase enzyme and 8 uL of total RNA input (min. 1000 ng). Such a prepared sample was incubated for 2 min at 37° C. followed by addition of 1 uL of 100 mM of DTT to deactivate the enzyme (final volume 11 uL) and incubated for 5 min at 55° C. The next step involved annealing of the gene specific reverse transcription primer (2 uM conc.), addition of 10 mM dNTPs mix (10 mM each) to 11 uL of RNA from previous step. The reaction was to heat up for 5 min at 65° C. and immediately put on ice for at least 1 min. Subsequent step was processed by mixing a 5× SuperScript IV buffer with 100 mM DTT, Ribonucleotide Inhibitor, SuperScript IV RT enzyme (7 uL) and 13 uL of RNA with pre-annealed RT primer. Reverse transcription required incubation for 10 min at 55° C., followed by 10 min at 80° C. and final hold at 4 C for an infinite amount of time. Final step of the reverse transcription involved removal of RNA from the reaction with 1 uL of E. coli RNase H and 20 min incubation at 37° C. After cDNA synthesis, cDNA was diluted 5× with water. In the next step of PCR amplification

5× PrimeSTAR GXL Buffer, dNTP Mixture, forward and reverse primers (15 uM), PrimerSTAR GXL DNA Polymerase and water (total 40 uL), and 10 uL of diluted cDNA from the previous step was used (total volume 50 uL reaction). 4 PCR wells were used for each sample to increase the yield. The amplification reaction was carried out in a preheated thermocycler with the following conditions: pre-incubation at 98° C. for 1 min followed by 33× cycles at 98° C. for 20 sec, 60° C. for 15 sec, followed by incubation at 68° C. for 20 min. The reaction was stopped by lowering the temperature down to 4° C. for infinite time. Each amplicon/sample had 4 wells to be pooled together before being purified for the first time with AMPure® PB Beads (100-265-900, Pacific Biosciences) according to manufacturer protocol at 0.45× ratio. Quantity of the amplicons were measured with Qubit BR DNA assay while size of them was verified with TapeStation, Generated amplicons were subjected for a second purification with AMPure® PB Beads (100-265-900, Pacific Biosciences) according to manufacturer protocol at 3.1× ratio to remove potential <7 kb fragments left. After the second purification, quantity of the amplicons were measured with Qubit BR DNA assay while size of them was verified with TapeStation, Genomic DNA Screen Tapes.

DNA and cDNA amplicons from the same sample were normalized according to Qubit values and pooled in equimolar concentrations. Each sample pool was ligated with sequencing index adapters and loaded onto the sequencing flow cell according to the manufacturer protocol (Pacific Biosciences of California, Inc., Menlo Park, USA). Pooling of DNA and cDNA samples was performed to bin results from each sample into an indexed bulk data.

Primers for PCR of HTT Gene from Genomic DNA i.e. Exonic SNV+Intronic SNP

TABLE 1

| Name | Oligonucleotide Sequence | Number |
|---|---|---|
| PCR Fwd gDNA Primer | 5′-GATGGATTCCTGTTTAAATGCCGT-3′ | SEQ ID NO: 1 |
| PCR Rev gDNA Primer | 5′-GGCTAATGACCCTTGTTGAGGA-3′ | SEQ ID NO: 2 |

Primers for RT-PCR of HTT Gene from mRNA/cDNA i.e. CAG Repeats+Exonic SNV

TABLE 2

| Name | Oligonucleotide Sequence | Number |
|---|---|---|
| RT Gene Specific | 5′-ATGCTGGGCTCTGTCACTAA-3′ | SEQ ID NO: 3 |
| Primer | | |
| PCR Fwd Primer | 5′-AGCTGATGAAGGCCTTCGAGTCCCTCAAGTC-3′ | SEQ ID NO: 4 |
| PCR Rev Primer | 5′-GTGTTCCCAAAGCCTGCTCACGGCAC-3 | SEQ ID NO: 5 |

### Example 2

### Bioinformatic Analysis

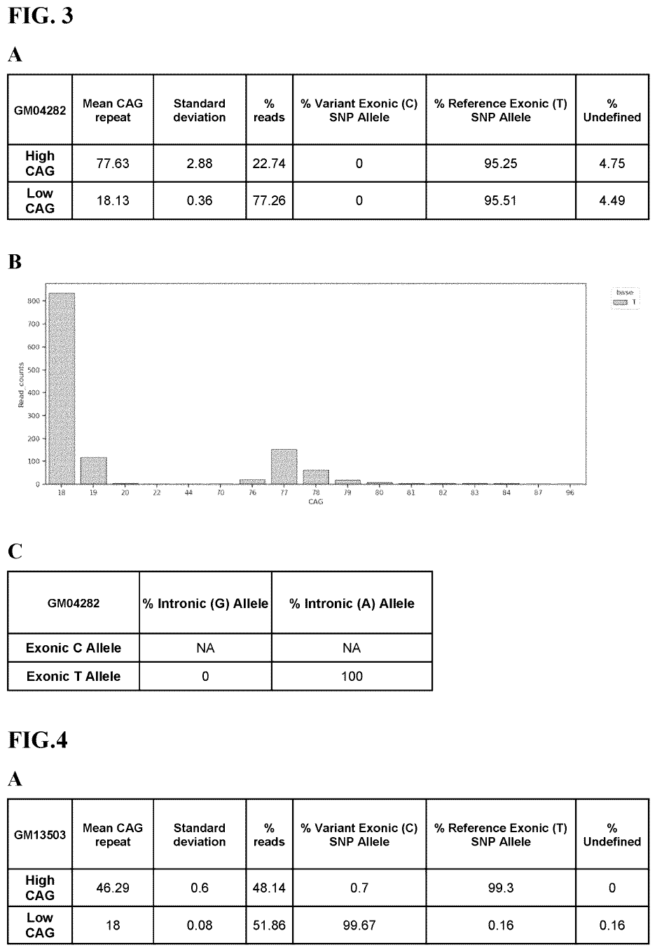

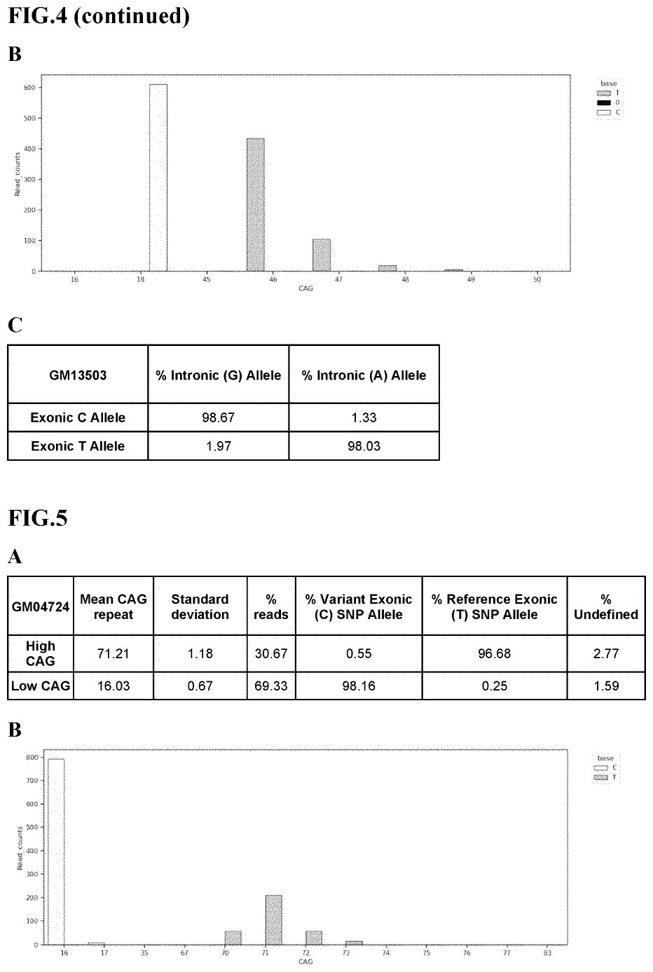

Both amplicon readouts (i.e., DNA sequence derived from the genomic DNA amplification and sequencing steps; and cDNA sequence derived from mRNA reverse transcription, amplification and sequencing steps) are initially aligned to the reference gene or complete human genome and processed with an open-source program to quantify the number of tandem repeats per molecule and extract nucleotide signal at desired SNP position. Alignment programs used include BWA, STAR or minimap2, while programs for quantification of CAG's include Tandem Repeat Genotyper, RepeatAnalysisTools or DeepRepeat software. Example results are shown for 3 different Coriell samples (The cell lines GM04282, GM13503 and GM04724 were obtained from NIGMS Human Genetic Cell Repository at the Coriell Institute for Medical Research) in FIGS. 3-5. Results of the cDNA analysis are shown in panel A (FIGS. 3-5) and visualized in panel B (FIGS. 3-5), illustrating which exonic SNP allele is phase with the high and low CAG repeat number and showing technical details such as mean CAG number, standard deviation, percent of reads containing high or low number of tandem repeats etc., Results of the DNA amplicon are shown in panel C (FIGS. 3-5) and indicate percentage of reads containing intronic polymorphism (i.e. Guanine or Adenine) against exonic polymorphism (i.e. Cytosine or Thymine). Herein, FIG. 3 describing the results for the sample GM04282 shows that based on the cDNA data that the mutant HTT allele with ~77.6 CAGs (“High CAG”) comprising ~22.7% of the reads, shows a phasing with the exonic T allele in ~95% of the reads. While the wild-type HTT allele with ~18.1 CAG's (“Low CAG”) comprising ~77.2% of reads also shows ~95% of the reads having exonic T allele. Both alleles have >4% of reads with undefined nucleotides at the position of the exonic reference SNP, which may be due to errors such as insertions or deletions. DNA amplicon data (FIG. 3C) shows that both the exonic and intronic SNPs are homozygous, and therefore this patient would not be suitable for allele-specific treatment. FIGS. 4C and 5C show the data for the samples GM13503 and GM04742 that are heterozygous for both exonic and intronic SNPs, which would make both of these individuals eligible for the allele-specific approach.

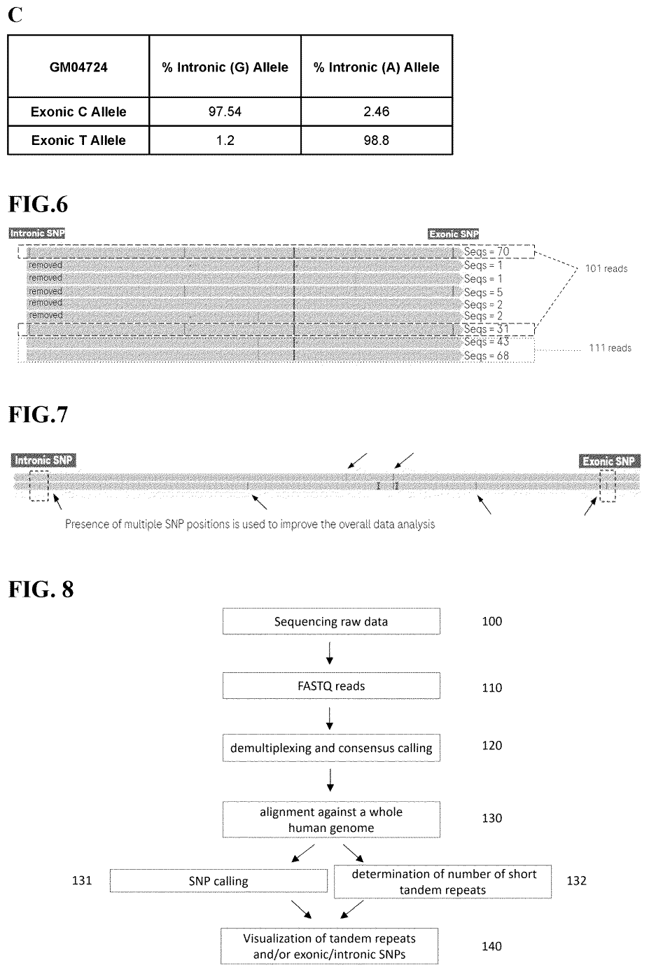

Hybrid analysis of the DNA and cDNA amplicon sequences, which is required for phasing of the CAG tandem repeats with the intronic SNP is based on tiling of the reads via exonic reference SNP—results of the each amplicon allowing this are shown in FIGS. 3,4 and 5 (A and C). Binning of data and filtration step are based on sequence similarity (i.e. 100%) represented in FIG. 6, where error prone reads based on the QC metrics are discarded while the signal with highest amount of readouts is consensus called. Individual haplotypes can be visualized with IGV software as indicated for the genomic DNA amplicon in FIG. 7 or bar plots in panel B (FIGS. 3-5)

### REFERENCES

Bečanović, K., Nørremølle, A., Neal, S. J., Kay, C., Collins, J. A., Arenillas, D., Lilja, T., Gaudenzi, G., Manoharan, S., Doty, C. N. and Beck, J. (2015) A SNP in the HTT promoter alters NF-κB binding and is a bidirectional genetic modifier of Huntington disease. Nature neuroscience, 18(6), pp. 807-816.
Carroll, J. B., Warby, S. C., Southwell, A. L., Doty, C. N., Greenlee, S., Skotte, N., Hung, G., Bennett, C. F., Freier, S. M. and Hayden, M. R. (2011) Potent and selective antisense oligonucleotides targeting single-nucleotide polymorphisms in the Huntington disease gene/allele-specific silencing of mutant huntingtin. Molecular Therapy, 19(12), pp. 2178-2185.
Claassen D. O., Corey-Bloom J., Dorsey E. R., Edmondson M., Kostyk S. K., LeDoux M. S., Reilmann R., Rosas H. D., Walker F., Wheelock V., Svrzikapa N., Longo K. A., Goyal J., Hung S., Panzara M. A. (2020) Genotyping single nucleotide polymorphisms for allele-selective therapy in Huntington disease. Neurol Genet, 6 (3) e430.
Flower M., Lomeikaite V., Ciosi M., Cumming S., Morales F., Lo K., Moss D. H., Jones L., Holmans P., Monckton D. G., Tabrizi S. J., (2019) MSH3 modifies somatic instability and disease severity in Huntington's and myotonic dystrophy type 1, Brain, 142(7):1876-1886
Goold R., Flower M., Moss D. H., Medway C., Wood-Kaczmar A., Andre R., Farshim P., Bates G. P., Holmans P., Jones L., Tabrizi S. J., (2019) FAN1 modifies Huntington's disease progression by stabilizing the expanded HTT CAG repeat, Human Molecular Genetics, 28(4):650-661.
Hannan, A. (2018) Tandem repeats mediating genetic plasticity in health and disease. Nat Rev Genet 19, 286-298.
Kartsaki, E., Spanaki, C., Tzagournissakis, M., Petsakou, A., Moschonas, N., MacDonald, M., & Plaitakis, A. (2006) Late-onset and typical Huntington disease families from Crete have distinct genetic origins. International Journal of Molecular Medicine, 17, 335-346.
Kay, C., Collins, J. A., Skotte, N. H., Southwell, A. L., Warby, S. C., Caron, N. S., Doty, C. N., Nguyen, B., Griguoli, A., Ross, C. J. and Squitieri, F. (2015) Huntingtin haplotypes provide prioritized target panels for allele-specific silencing in Huntington disease patients of European ancestry. Molecular Therapy, 23(11):1759-1771.
Lee, J. M., Gillis, T., Mysore, J. S., Ramos, E. M., Myers, R. H., Hayden, M. R., Morrison, P. J., Nance, M., Ross, C. A., Margolis, R. L. and Squitieri, F. (2012) Common SNP-based haplotype analysis of the 4p16. 3 Huntington disease gene region. The American Journal of Human Genetics, 90(3):434-444.
Ramos, E. M., Latourelle, J. C., Lee, J H. et al. (2012) Population stratification may bias analysis of PGC-1α as a modifier of age at Huntington disease motor onset. Hum Genet 131:1833-1840.
Shin, J. W., Shin, A., Park, S. S. and Lee, J. M. (2022) Haplotype-specific insertion-deletion variations for allele-specific targeting in Huntington's disease. Molecular Therapy—Methods & Clinical Development, 25:84-95.
Skotte, N. H., Southwell, A. L., Østergaard, M. E., Carroll, J. B., Warby, S. C., Doty, C. N., Petoukhov, E., Vaid, K., Kordasiewicz, H., Watt, A. T. and Freier, S. M. (2014) Allele-specific suppression of mutant huntingtin using antisense oligonucleotides: providing a therapeutic option for all Huntington disease patients. PloS one, 9(9), p.e107434.
Slatko B. E., Gardner A. F., Ausubel F. M. (2018) Overview of Next-Generation Sequencing Technologies. Curr Protoc Mol Biol. 122(1):e59.
Warby, S. C., Montpetit, A., Hayden, A. R., Carroll, J. B., Butland, S. L., Visscher, H., Collins, J. A., Semaka, A., Hudson, T. J. and Hayden, M. R. (2009) CAG expansion in the Huntington disease gene is associated with a specific and targetable predisposing haplogroup. The American Journal of Human Genetics, 84(3):351-366.

1. A method of phasing (i) at least one distant single nucleotide polymorphism of interest and (ii) short tandem repeats and/or at least one exonic single nucleotide polymorphism of interest within the same target gene locus of nucleic acids isolated from a biological sample, the method comprising:
a. performing an amplification step comprising contacting genomic DNA isolated from said biological sample comprising the target gene locus with a first set of oligonucleotide primers to produce a first amplification product comprising the at least one distant single nucleotide polymorphism of interest and at least one exonic reference single nucleotide polymorphism in the same target gene locus;
b. performing a reverse transcription and amplification step comprising contacting mRNA isolated from said biological sample comprising the target gene locus with a second set of oligonucleotide primers to produce a second amplification product comprising the short tandem repeats and/or the at least one exonic single nucleotide polymorphism of interest and said at least one exonic reference single nucleotide polymorphism;
c. determining nucleic acid sequences of the first and the second amplification product;
d. aligning the nucleic acid sequences of the first and the second amplification product determined in step (c) based on a position of the at least one exonic reference single nucleotide polymorphism; and
e. determining a haplotype of (i) the at least one distant single nucleotide polymorphism of interest and (ii) the short tandem repeats and/or the at least one exonic single nucleotide polymorphism of interest in the sample.

2. (canceled)

3. The method of claim 1, wherein the at least one distant single nucleotide polymorphism of interest is an intronic single nucleotide polymorphism.

4. The method of claim 3, wherein the intron carrying the at least one distant single nucleotide polymorphism of interest is located adjacent to the exon carrying the at least one exonic reference single nucleotide polymorphism within the same target gene locus of the isolated genomic DNA.

5. The method of claim 1, wherein the amplification step (a) and the reverse transcription and amplification step (b) are performed in separate reaction vessels.

6. The method of claim 1, wherein the nucleic acid sequences of the first and the second amplification product in step (c) are determined using long-read sequencing.

7. The method of claim 1, wherein the biological sample is selected from the group consisting of tissue, tumor tissue, blood, saliva and cell lines derived from an individual.

8. The method of claim 1, wherein prior to aligning the nucleic acid sequences of the first and the second amplification product in step (d) the method further comprises the step of aligning the nucleic acid sequences of the first and the second amplification product to a nucleic acid sequence of a reference gene of the target gene locus or to a nucleic acid sequence of a complete human genome or to parts thereof comprising a reference gene of the target gene locus.

9. The method of claim 1, wherein the target gene locus is a huntingtin gene.

10. The method of claim 9, wherein the short tandem repeats are CAG repeats.

11. The method of claim 1, wherein the at least one distant single nucleotide polymorphism of interest is selected from the group consisting of rs7685686, rs362331, rs363088, rs362273, rs2024115, rs6446723, rs363064, rs2285086, rs6844859, rs363080, rs34315806, rs2298967, rs362271, rs363099, rs3121419, and rs16843804.

12. An in vitro method for diagnosing if an individual has a risk of developing Huntington's disease, the method comprising:
a. determining the haplotype of the at least one distant single nucleotide polymorphism of interest and the short tandem repeats in an in vitro sample obtained from said individual using the method of claim 1; and
b. determining the individual's risk of developing Huntington's disease based on the haplotype of the at least one distant single nucleotide polymorphism of interest and the CAG short tandem repeats determined in step (a).

13. An in vitro method of identifying a patient having Huntington's disease as likely to respond to a therapy targeting the at least one single nucleotide polymorphism of interest, the method comprising:
a. determining the haplotype of the at least one distant single nucleotide polymorphism of interest and the short tandem repeats in an in vitro sample obtained from said patient using the method of claim 1; and
b. identifying the patient as likely to respond to the therapy based on the haplotype determined in step (a) and a determination of the presence of a specific allele of the at least one single nucleotide polymorphism of interest.

14. The method of claim 13, wherein the therapy targeting the at least one distant single nucleotide polymorphism of interest is an antisense oligonucleotide treatment directed to silence an RNA molecule comprising the specific allele of the at least one single nucleotide polymorphism of interest.

15. A kit for determining a nucleic acid sequence of (i) at least one distant single nucleotide polymorphism of interest and (ii) short tandem repeats and/or at least one exonic single nucleotide polymorphism of interest within the same target gene locus of nucleic acids isolated from a biological sample, the kit comprising:
a first set of oligonucleotide primers to produce a first amplification product comprising the at least one distant single nucleotide polymorphism of interest and at least one exonic reference single nucleotide polymorphism in the same target gene locus; and
a second set of oligonucleotide primers to produce a second amplification product comprising the short tandem repeats and/or the at least one exonic single nucleotide polymorphism of interest and said at least one exonic reference single nucleotide polymorphism.

16. An antisense oligonucleotide specifically hybridizing to the at least one distant single nucleotide polymorphism of interest in a huntingtin (HTT) gene for use for treating a patient having Huntington's disease, wherein the patient is selected for treatment when determining a specific haplotype of the at least one distant single nucleotide polymorphism of interest and the short tandem repeats detected in a biological sample of the patient, and wherein the specific haplotype is determined using the method of claim 1.

17. In vitro use of haplotype determination of the at least one distant single nucleotide polymorphism of interest and the short tandem repeats detected in the HTT gene determined in a biological sample of an individual for diagnosing Huntington's disease, wherein the detection of a specific haplotype of the at least one distant single nucleotide polymorphism of interest and the short tandem repeats detected in the biological sample of the individual indicates that the individual has Huntington's disease, and wherein the specific haplotype is determined using the method of claim 1.

18. In vitro use of haplotype determination of the at least one distant single nucleotide polymorphism of interest and the short tandem repeats detected in the HTT gene determined in a biological sample of a patient having Huntington's disease for determining the patient as likely to respond to a therapy comprising an antisense oligonucleotide specifically hybridizing to the at least one distant single nucleotide polymorphism of interest in the HTT gene, wherein the patient is identified as being more likely to respond to the therapy when a specific haplotype of the at least one distant single nucleotide polymorphism of interest and the short tandem repeats is detected in a biological sample of the patient, and wherein the specific haplotype is determined using the method of claim 1.


SEQUENCE LISTING (5 sequences)

SEQ ID NO: 1 - DNA, 24 bp, synthetic construct
  source 1..24: mol_type = other DNA
gatggattcctgtttaaatgccgt

SEQ ID NO: 2 - DNA, 22 bp, synthetic construct
  source 1..22: mol_type = other DNA
ggctaatgacccttgttgagga

SEQ ID NO: 3 - DNA, 20 bp, synthetic construct
  source 1..20: mol_type = other DNA
atgctgggctctgtcactaa

SEQ ID NO: 4 - DNA, 31 bp, synthetic construct
  source 1..31: mol_type = other DNA
agctgatgaaggccttcgagtccctcaagtc

SEQ ID NO: 5 - DNA, 26 bp, synthetic construct
  source 1..26: mol_type = other DNA
gtgttcccaaagcctgctcacggcac